(19) United States
(12) Patent Application Publication
(10) Pub. No.: US 2013/0323171 A1
(43) Pub. Date: Dec. 5, 2013

(54) RADIOLABELED BBN ANALOGS FOR PET IMAGING OF GASTRIN-RELEASING PEPTIDE RECEPTORS

(71) Applicant: The Board of Trustees of the Leland Stanford Junior University, Palo Alto, CA (US)

(72) Inventors: Zhen Cheng, Mountain View, CA (US); Hongguang Liu, Chaoyang (CN); Xiang Hu, Chang De (CN); Yang Liu, Hangzhou (CN)

(21) Appl. No.: 13/910,318

(22) Filed: Jun. 5, 2013

Related U.S. Application Data
(60) Provisional application No. 61/655,621, filed on Jun. 5, 2012.

(51) Int. Cl. C07K 7/08; G01N 33/50; A61K 51/08

(52) U.S. Cl. CPC C07K 7/086; A61K 51/088; G01N 33/5091
USPC 424/1.69; 530/326; 435/7.23

(57) ABSTRACT

Radiolabeled bombesin (BBN) analogs that bind to the gastrin-releasing peptide receptor (GRPR) represent a topic of active investigation for the development of molecular probes for positron emission tomography (PET) or single-photon emission computed tomography (SPECT) of prostate cancer (PCa). RM1 and AMBA have been identified as the two most promising BBN peptides for GRPR-targeted cancer imaging and therapy. In this study, to develop a clinically translatable BBN-based PET probe, we synthesized and evaluated ¹⁸F—AlF- and ⁶⁴Cu-radiolabeled RM1 and AMBA analogs for their potential application in PET imaging of PCa.

### CROSS-REFERENCE TO RELATED APPLICATION

This application claims priority to U.S. Provisional Patent Application Ser. No. 61/655,621 entitled “RADIOLABELED BBN ANALOG FOR PET IMAGING OF GASTRIN-RELEASING PEPTIDE RECEPTORS” which is hereby incorporated by reference in its entirety.

### STATEMENT REGARDING FEDERALLY SPONSORED RESEARCH OR DEVELOPMENT

This invention was made with Government support under contracts DOD-PCRP-NIA PC094646 awarded by the Department of Defense and NCI 5R01CA119053 awarded by the National Institutes of Health. The Government has certain rights in this invention.

### TECHNICAL FIELD OF THE DISCLOSURE

The present disclosure relates to delivery of radiolabeled and other ligands to a cell or tissue. The disclosure further relates to methods of ligand delivery to, and imaging of, cells and tissues expressing a gastrin-releasing peptide receptor, in particular prostate cancer cells.

### BACKGROUND

Prostate cancer (PCa) is the second leading cause of cancer death in men in the United States (Siegel et al., (2012) CA Cancer J. Clin. 62: 10-29). It is remains crucial to develop novel imaging probes and techniques for the primary diagnosis, follow-up, and monitoring of the recurrence of PCa. The clinical evaluation of certain positron emission tomography (PET) probes, such as ¹⁸F-fluorodeoxyglucose (¹⁸F-FDG), radiolabeled acetate and choline, has highlighted the pivotal role that PET might play in the imaging and characterization of PCa patients. The discovery of novel PET molecular probes is expected to dramatically facilitate the diagnosis, prognosis and stratification of PCa patients for effective therapeutic regimens (Jana & Blaufox (2006) Seminars Nucl. Med. 36: 51-72; Bouchelouche & Oehr (2008) J. Urol. 179: 34-45; Larson & Schoder (2008) Curr. Opin. Urol. 18: 65-70; Oehr & Bouchelouche (2007) Curr. Opin. Oncol. 19: 259-264; Schoder & Larson (2004) Semin. Nucl. Med. 34: 274-292; Delgado et al., (2009) Actas Urol. Esp. 33: 11-23).

The gastrin-releasing peptide receptor (GRPR) is over-expressed in many human epithelial malignancies, including PCa (Markwalder & Reubi (1999) Cancer Res. 59: 1152-1159; Jensen et al., (2008) Pharmacol. Rev. 60:1-42), breast cancer (Gugger & Reubi (1999) Am. J. Pathol. 155: 2067-2076) and lung cancer (Bostwick et al., (1984) Am. J. Pathol. 117: 195-200). GRPR-binding ligands represent potential diagnostic and therapeutic agents for targeting of GRPR-positive tumors. Examples of such GRPR-binding ligands include mammalian gastrin-releasing peptide (GRP) and its amphibian homolog bombesin (BBN) (Johnson et al., (2006) Cancer Biotherapy Radiopharmaceut. 21: 155-166).

These two peptides share homology in a C-terminal region, BBN(7-14), which is composed of the following eight amino acid residues: Gln-Trp-Ala-Val-Gly-His-Leu-Met-NH2. BBN(7-14) has been extensively used for the development of molecular probes for the imaging of PCa (Smith et al., (2005) Nucl. Med. Biol. 32: 733-740; Ananias et al., (2008) Curr. Pharm. Des. 14: 3033-3047; Abd-Elgaliel et al., (2008) Bioconjugate Chem. 19: 2040-2048; Garrison et al. (2008) Bioconjugate Chem. 19: 1803-1812; Prasanphanich et al., (2007) Proc. Natl. Acad. Sci. USA. 104: 12462-12467; Xu et al., (2012) ACS Macro Lett. 1: 753-757). Moreover, a variety of BBN analogs, including both agonists and antagonists, have been radiolabeled with different radionuclides for GRPR-targeted PCa imaging and therapy (Graham & Menda (2311) J. Nucl. Med. 52(Suppl 2): 56S-63S; Ambrosini et al., (2011) J. Nucl. Med. 52(Suppl 2): 42S-55S; Ait-Mohand et al., (2011) Bioconjug Chem. 22: 1729-1735; Lantry et al., (2006) J. Nucl. Med. 47: 1144-1152; Mansi et al., (2009) Clin Cancer Res. 15: 5240-5249; Schroeder et al., (2011) Eur. J. Nucl. Med. Mol. Imaging. 38: 1257-1266; Ho et al., (2011) J. Biomed. Biotechnol. 2011: 101497; Lane et al., (2010) Nucl. Med. Biol. 37: 751-761; Maddalena et al., (2009) J. Nucl. Med. 50: 2017-2024).

Among those GRPR-targeted peptides, RM1 (DOTA-CH2CO-G-4-aminobenzoyl-f-W-A-V-G-H-Sta-L-NH2, antagonist) (Mansi et al., (2009) Clin Cancer Res. 15: 5240-5249) and AMBA (DOTA-CH2CO-G-4-aminobenzoyl-Q-W-A-V-G-H-L-M-NH2, agonist) (Lantry et al., (2006) J. Nucl. Med. 47: 1144-1152) have been shown to be two of the most promising candidates for PCa theranostics. These two peptides have been radiolabeled with radiometals (¹¹¹In, ⁶⁸Ga and ⁶⁴Cu) for single-photon emission-computed tomography (SPECT) and PET imaging of PCa (Mansi et al., (2009) Clin Cancer Res. 15: 5240-5249; Schroeder et al., (2011) Eur. J. Nucl. Med. Mol. Imaging. 38: 1257-1266; Ho et al., (2011) J. Biomed. Biotechnol. 2011: 101497; Lane et al., (2010) Nucl. Med. Biol. 37: 751-761). AMBA has also been radiolabeled with 177Lu for radionuclide therapy of PCa (Lantry et al., (2006) J. Nucl. Med. 47: 1144-1152; Maddalena et al., (2009) J. Nucl. Med. 50: 2017-2024). All of these radiocomplexes exhibit efficient tumor accumulation and favorable in vivo properties, which support their potential clinical application. More interestingly, ⁶⁸Ga-AMBA-based PET imaging has recently been found to be superior to metabolism-based imaging using ¹⁸F-methylcholine for scintigraphy of PCa (Schroeder et al., (2011) Eur. J. Nucl. Med. Mol. Imaging. 38: 1257-1266). These studies clearly suggest that the increased likelihood of successfully translating a BBN probe into clinic can be achieved using AMBA and RM1 scaffolds.

### SUMMARY

The present disclosure encompasses compositions and methods for PET-based detectable of the gastrin-releasing peptide receptor in tumors. In particular the disclosure provides detectably labeled probes wherein a radionuclide such as, but not limited to, the fluoride ion is attached to aluminum that is complexed to a chelating agent.

One aspect of the disclosure, therefore, encompasses embodiments of a probe that can selectively bind to a mammalian gastrin-releasing peptide receptor (GRPR), the probe comprising a bombesin analogue selected from RM1 and AMBA, and the chelator 1,4,7-triazacyclononane, 1-glutaric acid-4,7-acetic acid (NODAGA) conjugated thereto, wherein the probe has a formula I or II:

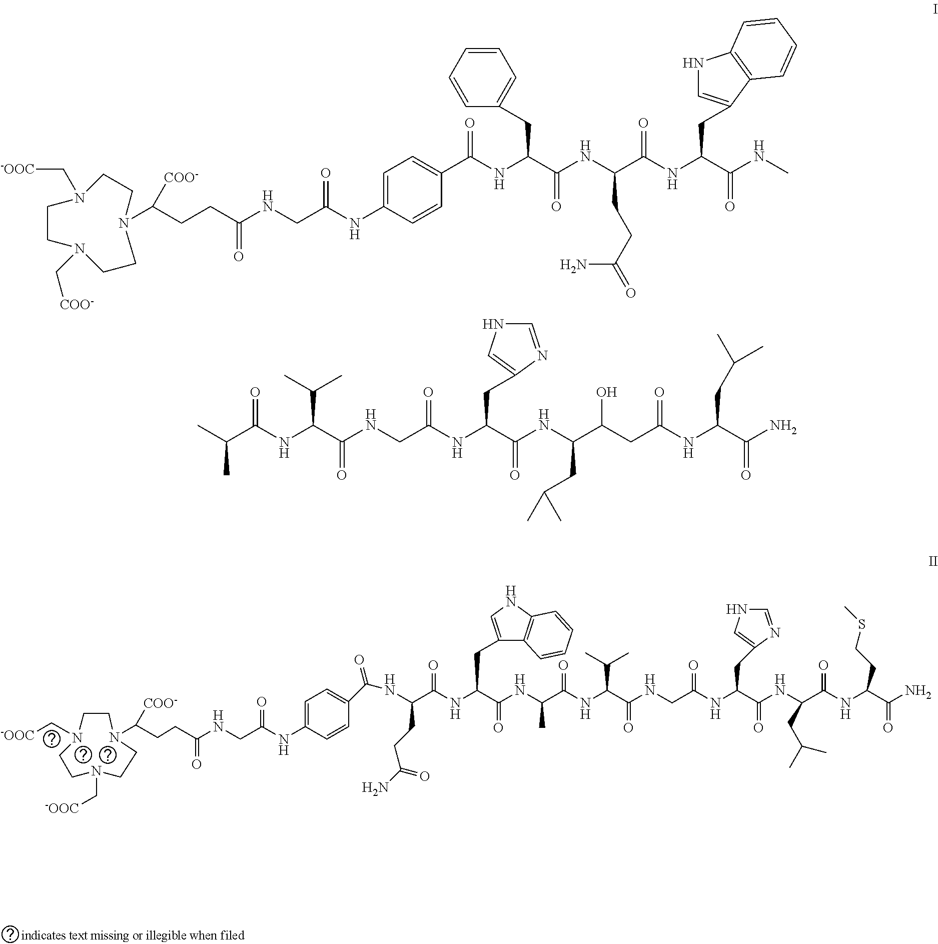

In embodiments of this aspect of the disclosure, the probe can further comprise a detectable label, wherein the detectable label can be ¹⁸F or ⁶⁴Cu.

In embodiments of this aspect of the disclosure, the probe can have a formula selected from the group consisting of formulas III, IV, V, and VI:

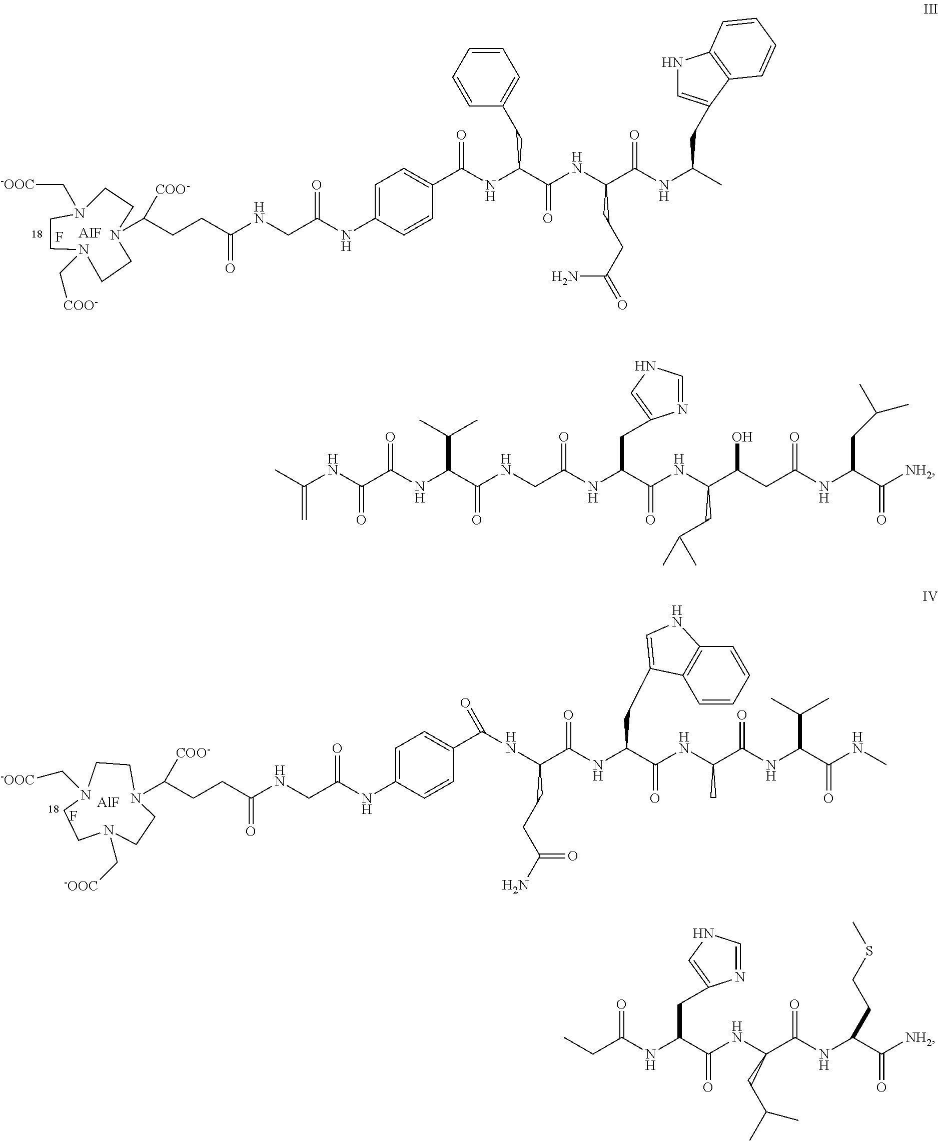

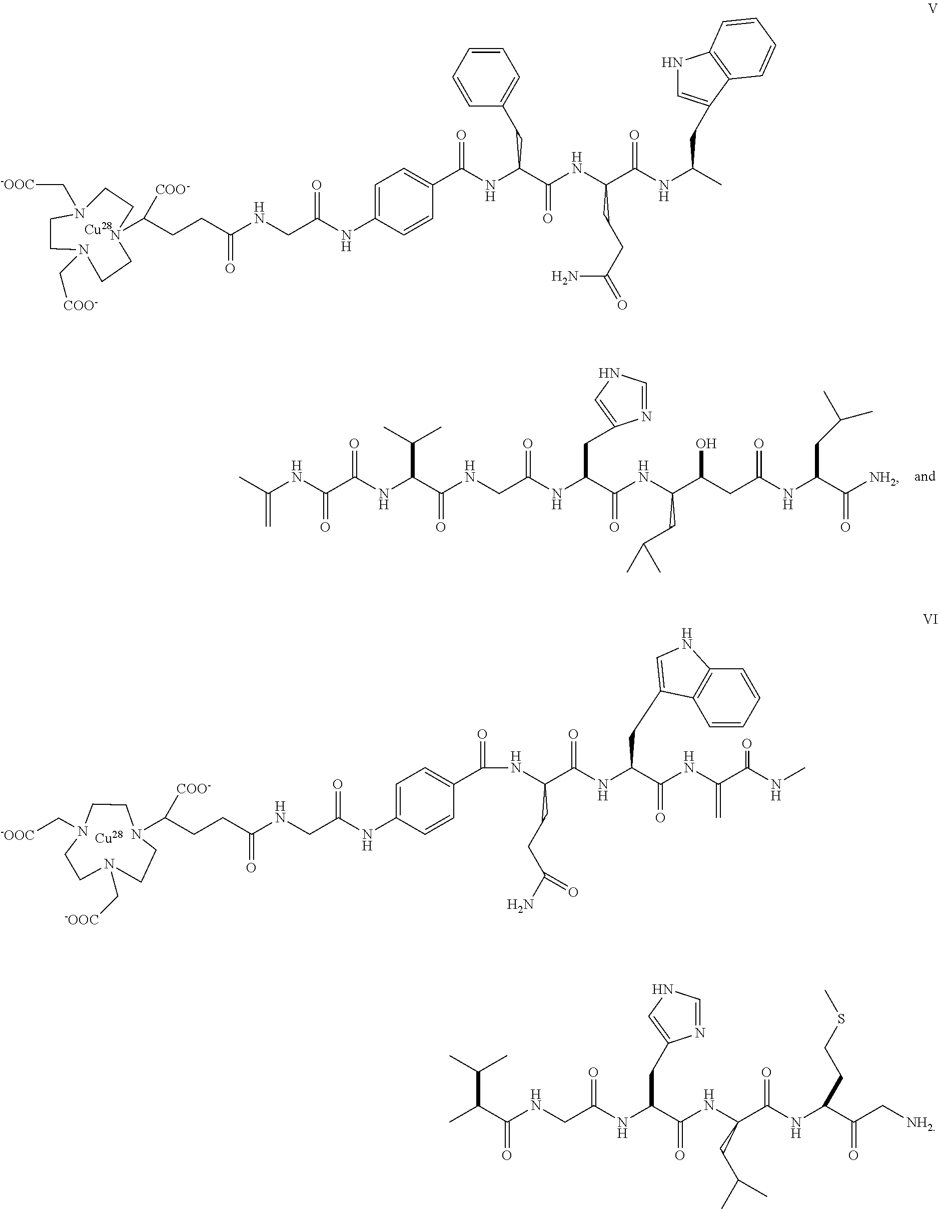

Another aspect of the disclosure encompasses embodiments of a pharmaceutically acceptable probe composition that can comprise at least one probe selected from the group consisting of from formulas III-VI, and a pharmaceutically acceptable carrier.

Yet another aspect of the disclosure encompasses embodiments of a method of identifying a cell or a population of cells expressing a gastrin-releasing peptide receptor, said method comprising: contacting a cell or population of cells with a composition that can comprise at least one probe having a radionuclide and selected from the group consisting of the formulas III-VI; allowing the probe to selectively bind to a gastrin-releasing peptide receptor of a cell or a population of cells; and detecting the radionuclide presence on the cell or population of cells, whereby the presence of the radionuclide indicates the cell or population of cells has a gastrin-releasing peptide receptor thereon.

In embodiments of this aspect of the disclosure, the probe composition can further comprise a pharmaceutically acceptable carrier.

In embodiments of this aspect of the disclosure, the method can further comprise the step of delivering the probe to a human or non-human animal.

Still another aspect of the disclosure encompasses embodiments of a method of detecting in a human or non-human animal a localized population of cancer cells having a gastrin-releasing peptide receptor, said method comprising the steps of: administering to a human or non-human animal a gastrin-releasing peptide receptor-specific probe selected from the group consisting of the formulas III-VI; determining the location of the probe in the recipient human or non-human animal; and identifying a tissue in the animal or human host, wherein the amount of the detectable label in the tissue is greater than in other tissues of the host, thereby identifying a population of cancer cells having a gastrin-releasing peptide receptor thereon.

In embodiments of this aspect of the disclosure, the gastrin-releasing peptide receptor-probe can be detected by positron emission tomography scanning.

Bombesin analogs are conjugated with NODA-GA-NHS, 2,2′-(7-(1-carboxy-4-((2,5-dioxopyrrolidin-1-yl)oxy)-4-oxobutyl)-1,4,7-triazonane-1,4-diyl)diacetic acid). The product is then radiolabeled with a positron emitter such as ¹⁸F, to form a PET imaging probe. The ability of this probe to image Gastrin-Releasing Peptide Receptors using micropositron emission tomography (microPET) was further evaluated on xenografted PC3 tumor mice models.

### BRIEF DESCRIPTION OF THE DRAWINGS

Many aspects of the disclosure can be better understood with reference to the following drawings.

FIG. 1A illustrates the structures of the probes NODAGA-RM1 (I) and NODAGA-AMBA (II).

FIG. 1B illustrates the structures of ¹⁸F—AlF-NODAGA-RM1 (III) and ¹⁸F—AlF-NODAGA-AMBA (IV).

FIG. 1C illustrates the structures of ⁶⁴Cu-NODAGA-RM1 (V) and ⁶⁴Cu-NODAGA-AMBA (VI).

FIG. 2 is a graph illustrating the inhibition of GRPR-binding by ¹²⁵I-[Tyr4]BBN on PC3 cells by RM1, NODAGA-RM1, AMBA and NODAGA-AMBA (n=4, mean±SD).

FIGS. 3A-3D shows a series of graphs illustrating the in vitro stability ⁶⁴Cu-NODAGA-RM1 (FIG. 3A), ⁶⁴Cu-NODAGA-AMBA (FIG. 3B), ¹⁸F—AlF-NODAGA-RM1 (FIG. 3C), and ¹⁸F—AlF-NODAGA-AMBA (FIG. 3D) in mouse serum after incubation at 37° C. for 1 h. Arrows indicate the intact probe.

FIG. 4A is a digital image illustrating the decay-corrected whole-body coronal small animal PET images of PC3 tumor-bearing male nude mice from a static scan at 0.5, 1.5 and 4 h after the injection of ⁶⁴Cu-NODAGA-AMBA. The tumors are indicated by arrows.

FIG. 4B is a graph illustrating small animal PET quantification of tumors and major organs at 0.5, 1.5 and 4 h after the injection of ⁶⁴Cu-NODAGA-AMBA.

FIG. 4C is a graph illustrating small animal PET quantification of tumors and major organs at 0.5, 1.5 and 4 h after the injection of ⁶⁴Cu-NOTAGA-RM1.

FIG. 4D is a graph illustrating the comparison of tumor-to-normal tissue (muscle, kidney and liver) ratios of ⁶⁴Cu-NODAGA-RM1 and ⁶⁴Cu-NODAGA-AMBA at 4 h p.i.

FIG. 5A is a digital image illustrating the decay-corrected whole-body coronal small animal PET images of PC3 tumor-bearing male nude mice from a static scan at 0.5, 1 and 2 h after the injection of ¹⁸F—AlF-NODAGA-AMBA without (top) or with (bottom) the co-injection of AMBA as a blocking agent (10 mg/kg body weight). The tumors are indicated by arrows.

FIG. 5B is a graph illustrating small animal PET image quantification of the tumors and the major organs at 0.5, 1 and 2 h after the injection of ¹⁸F—AlF-NODAGA-AMBA without AMBA as a blocking agent (10 mg/kg body weight).

FIG. 5C is a graph illustrating small animal PET image quantification of the tumors and the major organs at 0.5, 1 and 2 h after the injection of ¹⁸F—AlF-NODAGA-AMBA with (AMBA as a blocking agent (10 mg/kg body weight).

FIG. 5D is a graph illustrating a comparison of tumor-to-normal tissue (muscle, kidney and liver) ratios of ¹⁸F—AlF-NODAGA-AMBA without or with AMBA at 2 h p.i.

FIG. 6A is a digital image illustrating decay-corrected whole-body coronal small animal PET images of PC3 tumor-bearing male nude mice from a static scan at 0.5, 1 and 2 h after the injection of ¹⁸F—AlF-NODAGA-RM1 without (top) or with (bottom) AMBA as a blocking agent (10 mg/kg body weight). The tumors are indicated by arrows.

FIG. 6B is a graph illustrating small animal PET quantification of the tumors and the major organs at 0.5, 1 and 2 h after the injection of ¹⁸F—AlF-NODAGA-RM1 without AMBA as a blocking agent (10 mg/kg body weight).

FIG. 6C is a graph illustrating small animal PET quantification of the tumors and the major organs at 0.5, 1 and 2 h after the injection of ¹⁸F—AlF-NODAGA-RM1 with AMBA as a blocking agent (10 mg/kg body weight).

FIG. 6D is a graph illustrating a comparison of tumor-to-normal tissue (muscle, kidney and liver) ratios of ¹⁸F—AlF-NODAGA-RM1 without or with AMBA at 2 h p.i.

FIG. 7 illustrates the synthesis scheme for the probes of the disclosure.

FIG. 8 illustrates transition in the probe structure on association with an F—Al complex

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### Abbreviations

BBN, bombesin; GRPR, gastrin-releasing peptide receptor; GRP, gastrin-releasing peptide; PET, positron emission tomography; HPLC, high-performance liquid chromatography; p.i., post-injection.

### DEFINITIONS

In describing and claiming the disclosed subject matter, the following terminology will be used in accordance with the definitions set forth below.

The term “cell or population of cells” as used herein refers to an isolated cell or plurality of cells excised from a tissue or grown in vitro by tissue culture techniques. In the alternative, a population of cells may also be a plurality of cells in vivo in a tissue of an animal or human host and includes normal cells and can cancerous cells that are capable of forming a localized tumor in an animal.

The term “contacting a cell or population of cells” as used herein refers to delivering a composition such as, for example, a probe composition according to the present disclosure with or without a pharmaceutically or physiologically acceptable carrier to an isolated or cultured cell or population of cells, or administering the probe in a suitable pharmaceutically acceptable carrier to an animal or human host. Thereupon, it may be systemically delivered to the target and other tissues of the host, or delivered to a localized target area of the host. Administration may be, but is not limited to, intravenous delivery, intraperitoneal delivery, intramuscularly, subcutaneously or by any other method known in the art. One method is to deliver the composition directly into a blood vessel leading immediately into a target organ or tissue such as a prostate, thereby reducing dilution of the probe in the general circulatory system.

Imaging probes for use in the methods of the present disclosure may be labeled with one or more radioisotopes, preferably including, but not limited to, ¹¹C, ¹⁸F, ⁷⁶Br, ¹²³I, ¹²⁴I, or ¹³¹I, and are suitable for use in peripheral medical facilities and PET clinics. In particular embodiments, for example, the PET isotope can include, but is not limited to, ^{64/61}Cu, ¹⁸F, ¹¹¹In, and ⁶⁸Ga. Of particular advantage in the embodiments of the disclosure are radionuclides that are metallic and capable of binding to a chelating moiety conjugated to a GRP receptor ligand. Alternatively, a radionuclide such as ¹⁸F may be a component of a metallic salt, the anion of which may chelate with the probes of the disclosure, thereby including the cationic radionuclide in the probe composition. For example, but not intended to be limiting. ¹⁸F may be a component of aluminum fluoride, i.e. AlF₃, the metallic aluminum of which can chelate to the probes of the disclosure.

The term “peptide” as used herein refers to short polymers formed from the linking, in a defined order, of α-amino acids. The link between one amino acid residue and the next is known as an amide bond or a peptide bond. Proteins are polypeptide molecules (or consist of multiple polypeptide subunits). The distinction is that peptides are short and polypeptides/proteins are long. There are several different conventions to determine these. Peptide chains that are short enough to be made synthetically from the constituent amino acids are called peptides, rather than proteins, with one dividing line at about 50 amino acids in length.

Modifications and changes can be made in the structure of the peptides of this disclosure and still result in a molecule having similar characteristics as the peptide (e.g., a conservative amino acid substitution). For example, certain amino acids can be substituted for other amino acids in a sequence without appreciable loss of activity. Because it is the interactive capacity and nature of a peptide that defines that peptide's biological functional activity, certain amino acid sequence substitutions can be made in a peptide sequence and nevertheless obtain a peptide with like properties.

In making such changes, the hydropathic index of amino acids can be considered. The importance of the hydropathic amino acid index in conferring interactive biologic function on a peptide is generally understood in the art. It is known that certain amino acids can be substituted for other amino acids having a similar hydropathic index or score and still result in a peptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant peptide, which in turn defines the interaction of the peptide with other molecules, such as enzymes, substrates, receptors, antibodies, antigens, and the like. It is known in the art that an amino acid can be substituted by another amino acid having a similar hydropathic index and still obtain a functionally equivalent peptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent peptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent peptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a peptide as set forth above. In particular, embodiments of the peptides can include variants having about 50%, 60%, 70%, 80%, 90%, and 95% sequence identity to the peptide of interest.

The term “pharmaceutically acceptable carrier” as used herein refers to a diluent, adjuvant, excipient, or vehicle with which a heterodimeric probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the heterodimeric probes and pharmaceutically acceptable carriers preferably should be sterile. Water is a useful carrier when the heterodimeric probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “pharmaceutically acceptable” as used herein refers to a composition that, in contact with a cell, isolated from a natural source or in culture, or a tissue of a host, has no toxic effect on the cell or tissue.

The term “positron emission tomography” as used herein refers to a nuclear medicine imaging technique that produces a three-dimensional image or map of functional processes in the body. The system detects pairs of gamma rays emitted indirectly by a positron-emitting radioisotope, which is introduced into the body on a metabolically active molecule. Images of metabolic activity in space are then reconstructed by computer analysis. Using statistics collected from tens-of-thousands of coincidence events, a set of simultaneous equations for the total activity of each parcel of tissue can be solved by a number of techniques, and a map of radioactivities as a function of location for parcels or bits of tissue may be constructed and plotted. The resulting map shows the tissues in which the molecular probe has become concentrated. Radioisotopes used in PET scanning are typically isotopes with short half lives such as carbon-11 (about 20 min), nitrogen-13 (about 10 min), oxygen-15 (about 2 min), and fluorine-18 (about 110 min). PET technology can be used to trace the biologic pathway of any compound in living humans (and many other species as well), provided it can be radiolabeled with a PET isotope. The half life of fluorine-18 is long enough such that fluorine-18 labeled radiotracers can be manufactured commercially at an offsite location.

The terms “mammal” as used herein are used interchangeably and refer to an animal, preferably a warm-blooded animal such as a mammal. Mammal includes without limitation any members of the Mammalia including humans. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g., gorillas or chimpanzees), and rodents such as rats and mice.

The term “pharmaceutically acceptable carrier” as used herein refers to a diluent, adjuvant, excipient, or vehicle with which a therapeutic composition according to the disclosure is administered and which is approved by a regulatory agency of the federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like.

The term “cancer” as used herein shall be given its ordinary meaning and is a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer. However, it is understood that the compositions and methods of the disclosure are intended to target those cancer cells and tumors thereof that express the protein gastrin-releasing peptide receptor such as, but not limited to, a prostate cancer.

The term “probe” as used herein refers to a molecule that may selectively bind to a cell surface component such as the gastrin-releasing peptide receptor (GRPR) and not to other proteins or if so binding to non-GRPR proteins to a significantly lesser degree. It is contemplated that the probe molecule may be labeled with a detectable label, in particular a label that may be detected by PET.

The term “chelator” as used herein refers to a molecular moiety that may form ionic bonds to an anion and in particular metallic ions have at least two positive charges thereon.

The term “detectable label” as used herein refers to a molecule, atom, or ion, the presence of which may determined by the label emitting a signal such as an alpha particle, a gamma particle, and the like that may be received in such a manner as to provide to an observer an indication of the presence of the label.

The term “determining the location of the probe” as used herein refers to comparing a detectable signal from the probes of the disclosure when administered to a mammalian subject and overlaying the detectable signal therefrom with an outline or optical image of the subject, thereby identifying the position of the label within the subject relative to non-labeled regions thereof.

### Discussion

Molecular imaging of cancer is a fast growing research field. Molecular imaging technologies have demonstrated great benefits for better understanding cancer biology, as well as for facilitating cancer drug development and cancer early detection. Development of novel imaging methods and molecularly targeted probes, such as the probes of the disclosure, allow not only to locate a tumor, but also to visualize the expression and activity of specific molecular targets and biological processes in a tumor.

It has been learned that some cancer cells contain gastrin releasing peptide (GRP) receptors (GRP-R) of which there are a number of subtypes. In particular, it has been shown that several types of cancer cells have over-expressed or uniquely expressed GRP receptors. GRP and GRP analogues can selectively bind to the GRP receptor family. One especially useful GRP analogue is bombesin (BBN), a tetradecapeptide isolated from frog skin that can bind to GRP receptors with high specificity and with an affinity similar to GRP.

GRP receptors have been shown to be over-expressed or uniquely expressed on several types of cancer cells. In addition to being seen in prostate cancers, GRPR is also expressed in almost 60% of primary breast carcinoma cases and in almost all infiltrated lymph nodes. Extremely high numbers of GRPRs have also been detected in gastrointestinal stromal tumors. Functionally, binding of GRP receptor agonists (also autocrine factors) increases the rate of cell division of these cancer cells.

The fragments of bombesin useful in the embodiments of the present disclosure contain either the same primary structure of the bombesin GPR binding region, i.e. bombesin(7-14) or bombesin(8-14), or similar primary structures, with specific amino acid substitutions, that will specifically bind to GRP receptors. Compounds containing this bombesin GPR binding region (or binding moiety), when covalently linked to other groups may also be referred to as bombesin conjugates.

The disclosure provides bombesin analogs are conjugated with NODA-GA-NHS, 2,2′-(7-(1-carboxy-4-((2,5-dioxopyrrolidin-1-yl)oxy)-4-oxobutyl)-1,4,7-triazonane-1,4-diyl)diacetic acid). The GRP-selective probe product is then radiolabeled with a positron emitter such as ¹⁸F to form an imaging probe that may be detected by PET scanning. The ability of this probe to image gastrin-releasing peptide receptors using micropositron emission tomography (microPET) was further evaluated on xenografted PC3 tumor mice models and demonstrates that the probes are useful for administering to a mammalian subject to allow the detecting of a concentration of cells that express GPR receptors associated with a cancer cell, and in particular a prostate cancer.

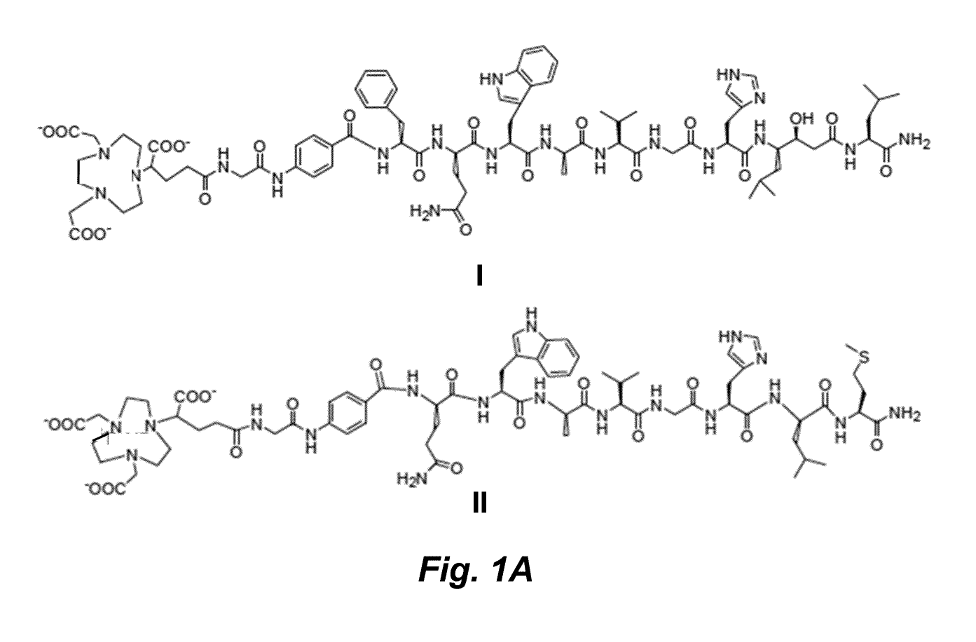

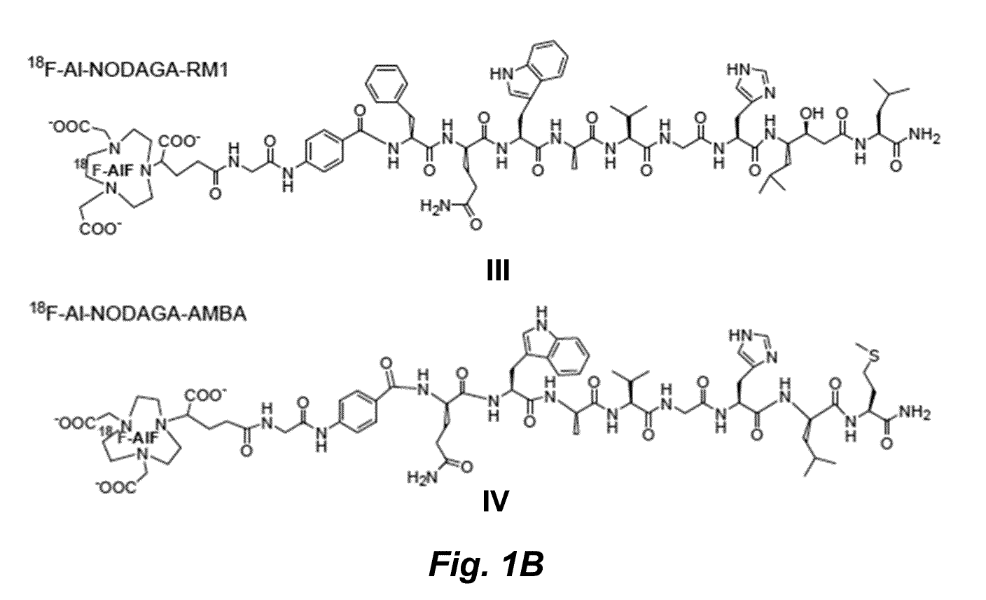

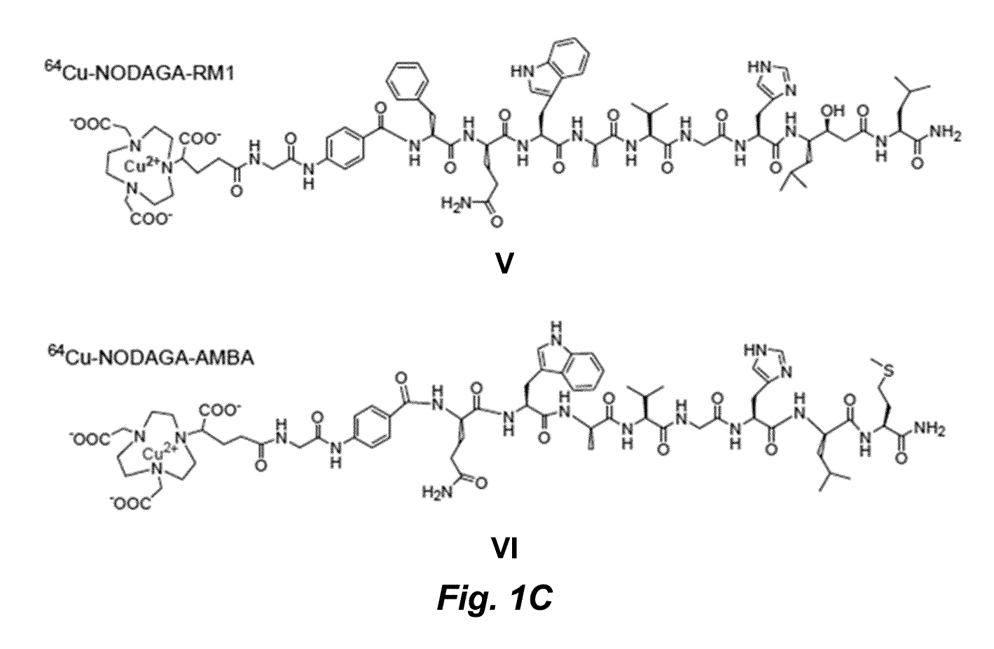

It is contemplated that the probes of the disclosure may incorporate any detectable metallic label including, but not limited to, ^{64/61} Cu, ⁸⁶Y, ⁸⁹Zr, and ⁶⁸Ga, or radioisotopes of Fe, Co, Ni, Cd, Cs, and the like that may chelate to the ODAGA-bombesin analogs of the disclosure. In the alternative, a PET-detectable label such as ¹⁸F may be attached to the GPR receptor-specific probes by first proving the ¹⁸F as a metallic fluoride, such as but not limited to, aluminum fluoride (AlF₃). In particular, it is recognized that ¹⁸F is the most important radionuclide for clinical PET and ⁶⁴Cu has also gained extensive interest in the context of PET probe development (Anderson & Ferdani (2009) Cancer Biotherapy Radiopharmaceut. 24: 379-393). The embodiments of the disclosure, therefore, encompasses the synthesis and use of 1,4,7-triazacyclononane, 1-glutaric acid-4,7-acetic acid (NODAGA)-conjugated RM1 and AMBA (NODAGA-RM1 (I), NODAGA-AMBA (II)) peptides that are shown as formulas I and II in FIG. 1A. These peptides may then be radiolabeled via a one-step chelation reaction such as with ¹⁸F—AlF or ⁶⁴Cu, in aqueous phase to generate ¹⁸F—AlF-NODAGA-RM1 (III), ¹⁸F—AlF-NODAGA-AMBA (IV), ⁶⁴Cu-NODAGA-RM1 (V) and ⁶⁴Cu-NODAGA-AMBA (VI), the structural formulas of which are shown in FIGS. 1B and 1C.

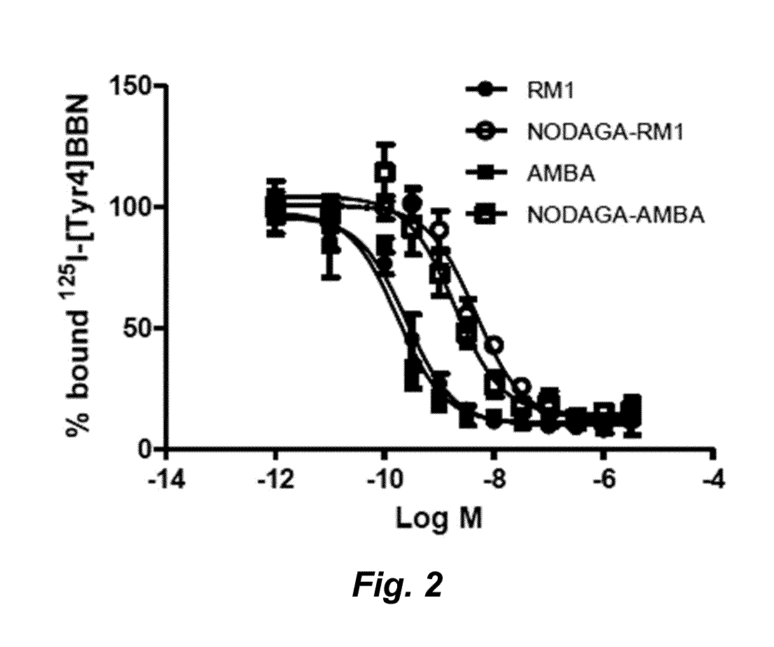

Accordingly, two BBN peptide analogs, AMBA and RM1, were labeled with ¹⁸F and used for PET imaging of PCa cells localized as a tumor. These two peptides were modified with the chelator NODAGA to render the bioconjugates more easily labeled with ⁶⁴Cu and ¹⁸F—AlF to form stable radiocomplexes. After modification with NODAGA, the IC50 of each of the conjugated peptides was still within the nM range, as shown in FIG. 2. Therefore, they were further radiolabeled with ⁶⁴Cu and ¹⁸F—AlF and tested in vivo.

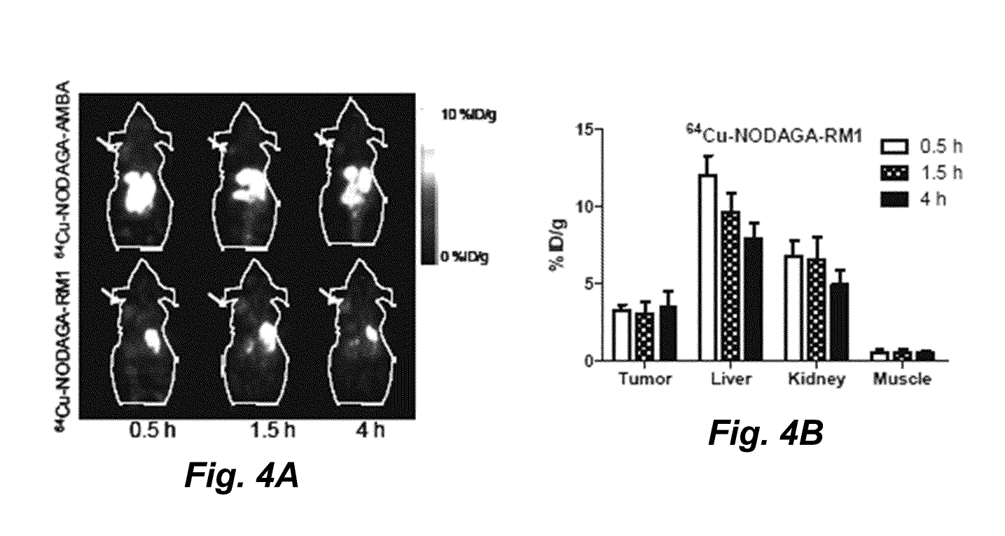

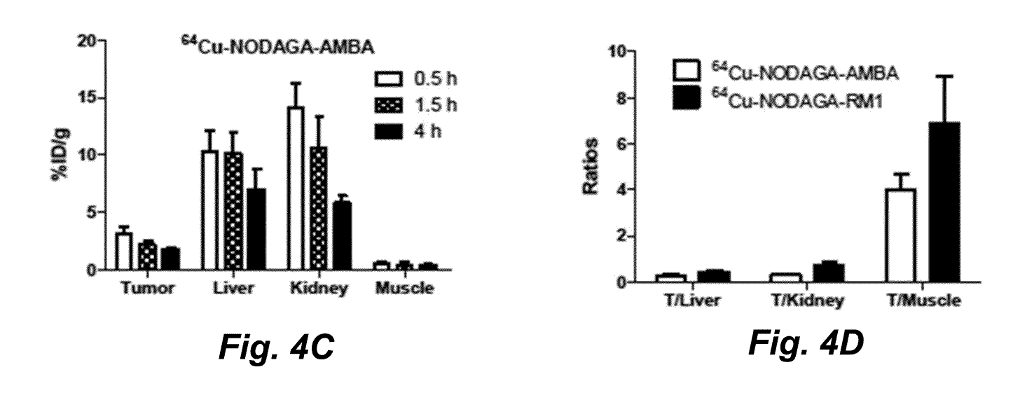

According to the in vitro serum stability assay and in vivo imaging results, both the ⁶⁴Cu- and ¹⁸F-labeled NODAGA-AMBA probes were less stable than the corresponding NODAGA-RM1 probes. Moreover, the ⁶⁴Cu- and ¹⁸F-labeled NODAGA-RM1 probes exhibited more favorable in vivo tumor retention and imaging quality compared with the ⁶⁴Cu- and ¹⁸F-labeled NODAGA-AMBA probes, as shown in FIGS. 4A-6D. BBN peptide antagonists and agonists exhibit different stabilities and in vivo behavior. It has been reported that the antagonist Demobesin-1 exhibits superior in vivo stability, increased tumor uptake and retention and faster pancreatic and renal clearance than the other four GRPR agonists (Schroeder Eur. J. Nucl. Med. Mol. Imaging. 37: 1386-1396). Mansi et al. also compared the performance of the radioantagonist [¹¹¹In]-RM1 with the radioagonist [¹¹¹In]-AMBA (Mansi et al., (2009) Clin Cancer Res. 15: 5240-5249). [¹¹¹In]-RM1 demonstrated relatively lower affinity for GRPR but more favorable pharmacokinetics and targeting properties, as supported by increased tumor uptake and tumor-to-normal tissue ratios. [¹¹¹In]-RM1 appeared to be superior to AMBA for in vivo SPECT imaging and for the targeted radiotherapy of GRPR-positive tumors. Consistent with these findings, the results herein using ⁶⁴Cu and ¹⁸F—AlF demonstrated that antagonist RM1-based PET probes exhibit significantly increased stability and more optimal imaging performance than the agonist AMBA-based probes and are, therefore, the most advantageous probes for use in the methods of the disclosure.

Recently, NOTA-8-Aoc-BBN(7-14)-NH₂ was labeled with ¹⁸F using the Al—¹⁸F method described by Dijkgraaf et al., and the results showed that the aluminum fluoride method does not affect the in vivo behavior of the peptide. For example, the uptake of ¹⁸F-labeled NOTA-8-Aoc-BBN(7-14)-NH₂ in the PC3 tumors was 2.15±0.55% ID/g, which is similar to the ⁶⁴Cu-labeled NOTA-8-Aoc-BBN(7-14) conjugate (3.59±0.70% ID/g) at 1 h p.i. (Prasanphanich et al., (2007) Proc. Natl. Acad. Sci. USA. 104: 12462-12467). The ¹⁸F—AlF-NODAGA-RM1 probe of the disclosure also exhibited similar uptake to ⁶⁴Cu-NODAGA-RM1 (4.6±1.5 vs. 3.3±0.38 at 0.5 h and 4.0±0.87 versus 3.0±0.76 at 1 h, respectively). More importantly, ¹⁸F—AlF-NODAGA-RM1 exhibited increased tumor uptake compared with ¹⁸F—AlF-NOTA-8-Aoc-BBN (7-14)-NH₂ (4.0±0.87 vs. 2.15±0.55), highlighting the advantages of using RM1 for PET probe development. Taken together, based on their favorable in vitro serum stability and in vivo tumor imaging properties (as shown in FIGS. 4A- and 6D), ⁶⁴Cu-NODAGA-RM1 and ¹⁸F—AlF-NODAGA-RM1 are the most advantageous for use in a clinical setting. In particular, the radioantagonist ¹⁸F—AlF-NODAGA-RM1 demonstrated simple radiosynthesis, i.e., the chelation of the metallic label to the probe takes place both rapidly and under mild conditions, increased tumor uptake and tumor-to-background contrast. ¹⁸F—AlF-NODAGA-RM1 is, therefore, especially useful for imaging PCa in clinical applications.

One aspect of the disclosure, therefore, encompasses embodiments of a probe that can selectively bind to a mammalian gastrin-releasing peptide receptor (GRPR), the probe comprising a bombesin analogue selected from RM1 and AMBA, and the chelator 1,4,7-triazacyclononane, 1-glutaric acid-4,7-acetic acid (NODAGA) conjugated thereto, wherein the probe has a formula I or II:

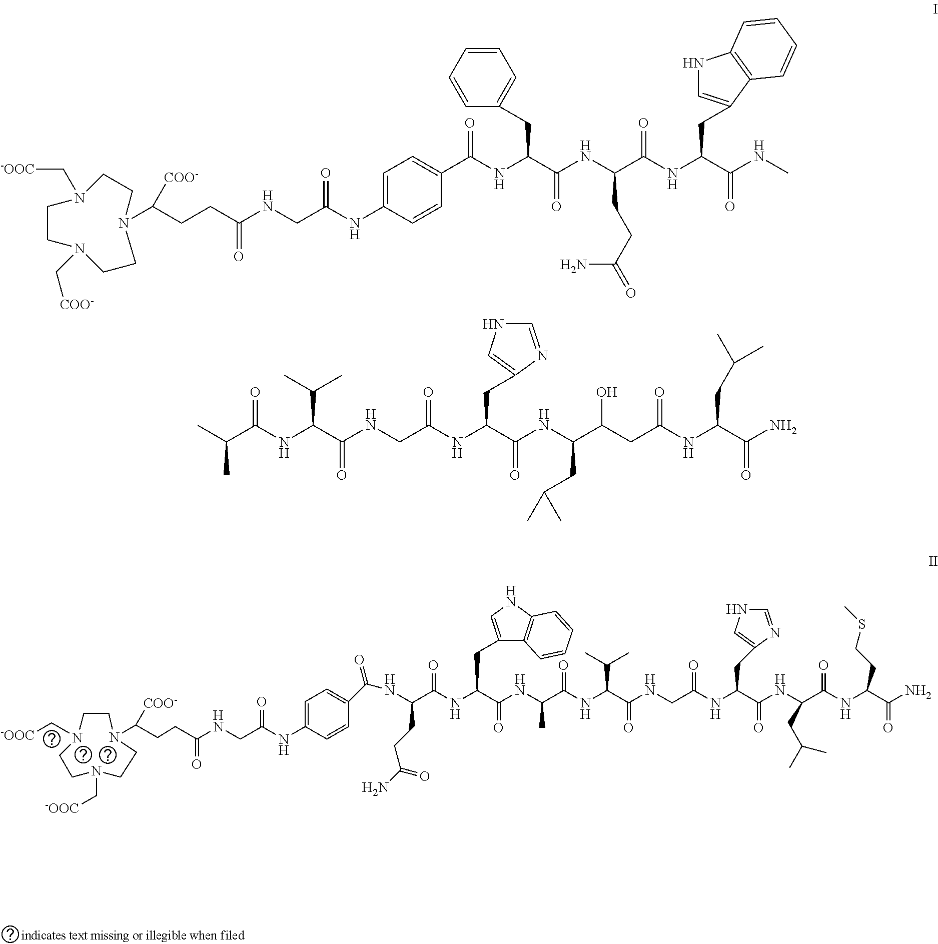

In embodiments of this aspect of the disclosure, the probe can further comprise a detectable label, wherein the detectable label can be ¹⁸F or ⁶⁴Cu.

In embodiments of this aspect of the disclosure, the probe can have a formula selected from the group consisting of formulas III, IV, V, and VI:

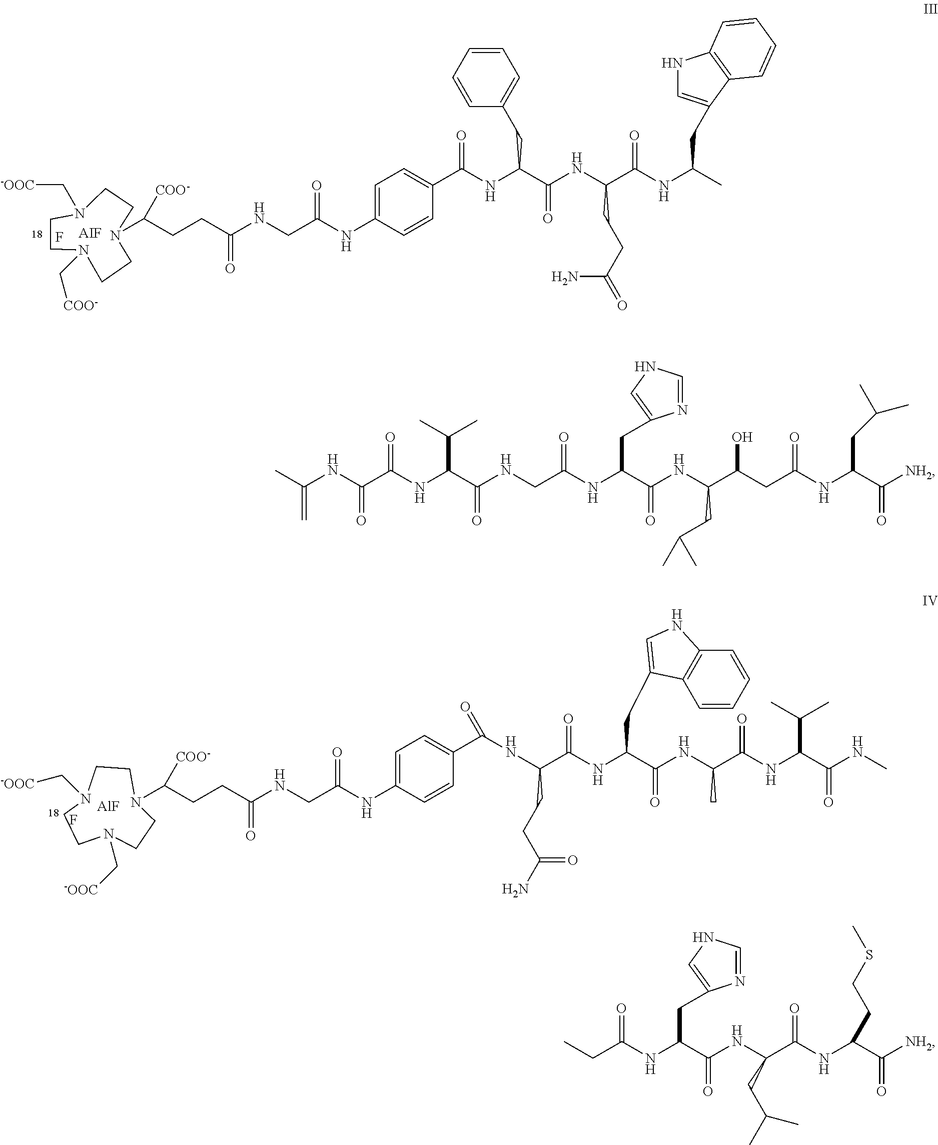

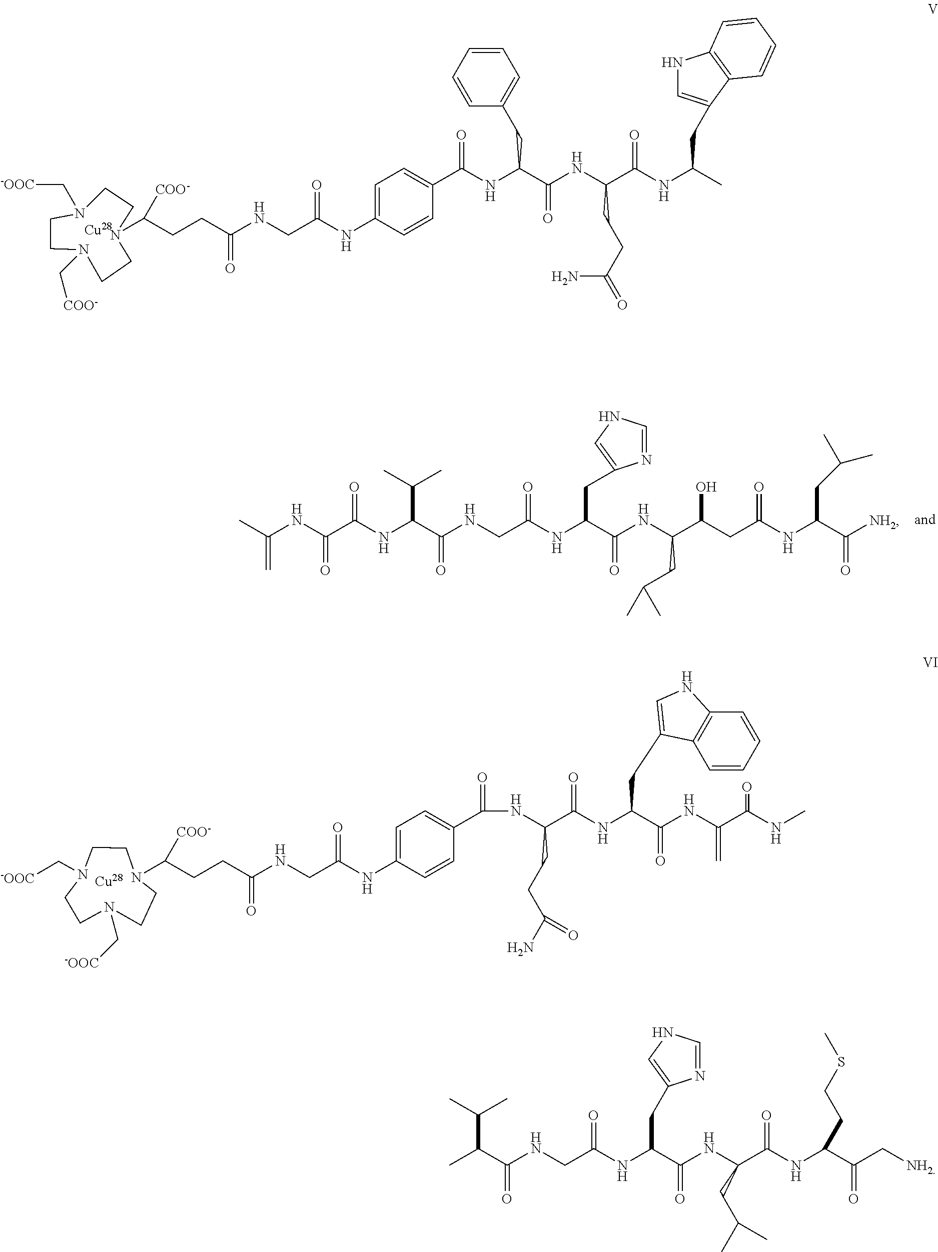

In embodiments of this aspect of the disclosure, the probe can have the formula III.

In embodiments of this aspect of the disclosure, the probe can have the formula IV.

In embodiments of this aspect of the disclosure, the probe can have the formula V.

In embodiments of this aspect of the disclosure, the probe can have the formula VI.

Another aspect of the disclosure encompasses embodiments of a pharmaceutically acceptable probe composition that can comprise at least one probe selected from the group consisting of from formulas III-VI:

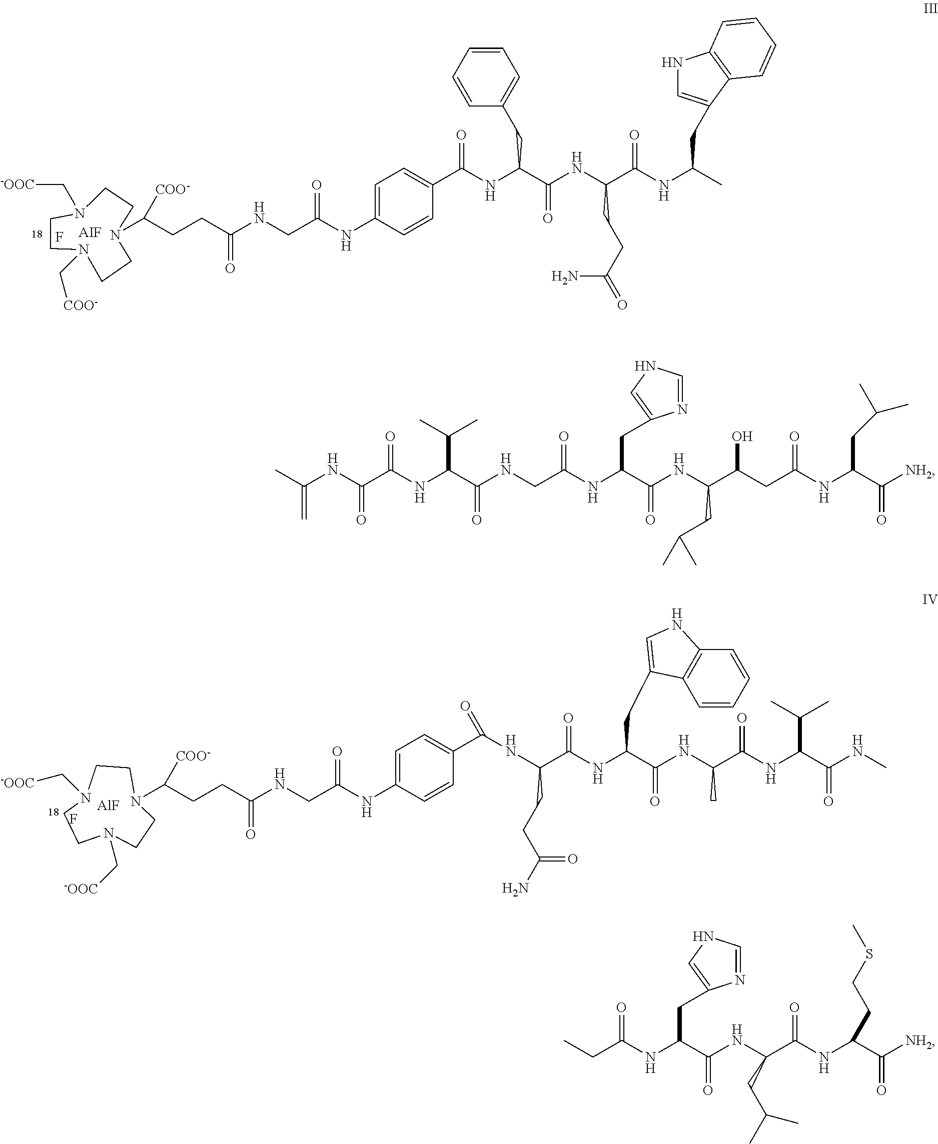

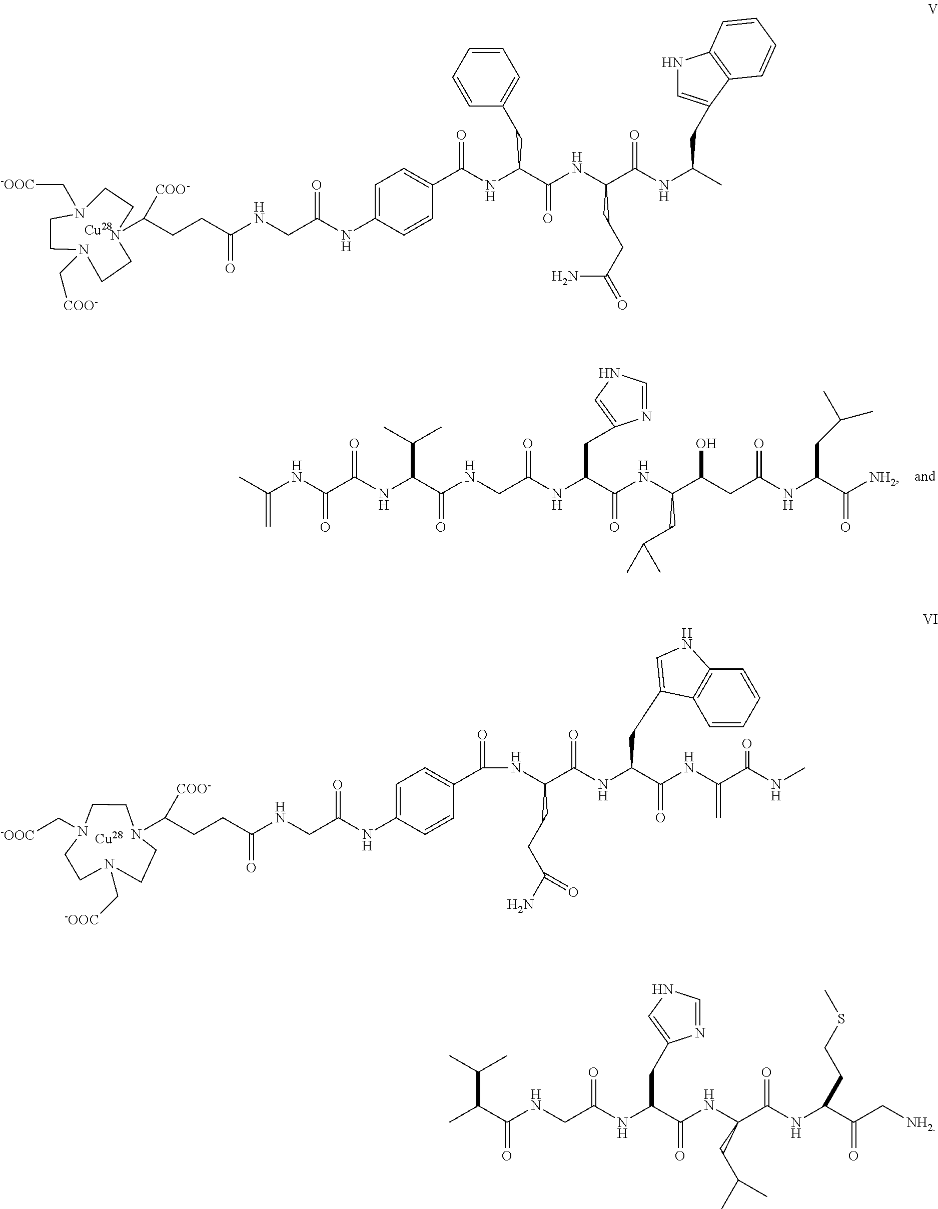

and a pharmaceutically acceptable carrier.

Yet another aspect of the disclosure encompasses embodiments of a method of identifying a cell or a population of cells expressing a gastrin-releasing peptide receptor, said method comprising: contacting a cell or population of cells with a composition that can comprise at least one probe having a radionuclide and selected from the group consisting of the formulas III-VI:

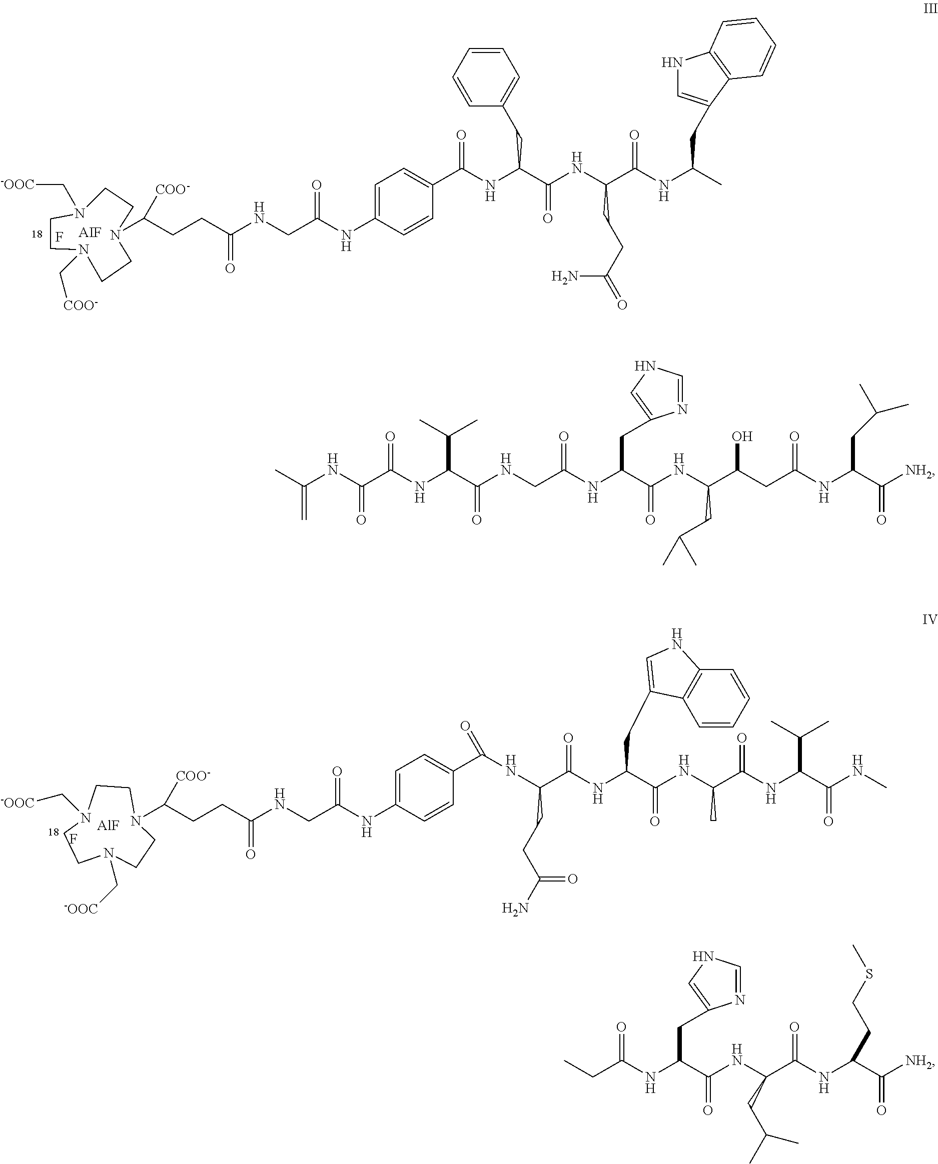

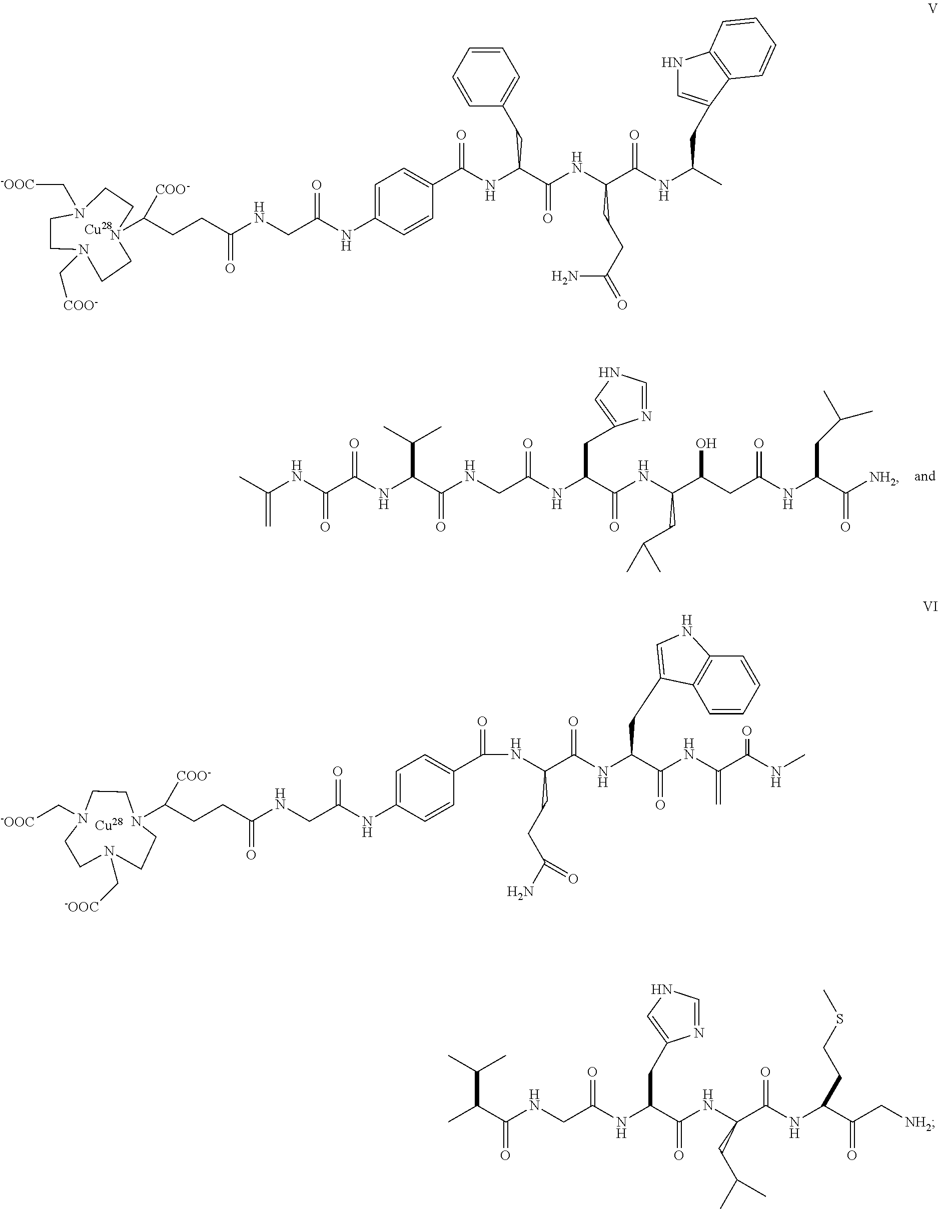

allowing the probe to selectively bind to a gastrin-releasing peptide receptor of a cell or a population of cells; and detecting the radionuclide presence on the cell or population of cells, whereby the presence of the radionuclide indicates the cell or population of cells has a gastrin-releasing peptide receptor thereon.

In embodiments of this aspect of the disclosure, the probe composition can further comprise a pharmaceutically acceptable carrier.

In embodiments of this aspect of the disclosure, the method can further comprise the step of delivering the probe to a human or non-human animal.

Still another aspect of the disclosure encompasses embodiments of a method of detecting in a human or non-human animal a localized population of cancer cells having a gastrin-releasing peptide receptor, said method comprising the steps of: administering to a human or non-human animal a gastrin-releasing peptide receptor-specific probe selected from the group consisting of the formulas III-VI:

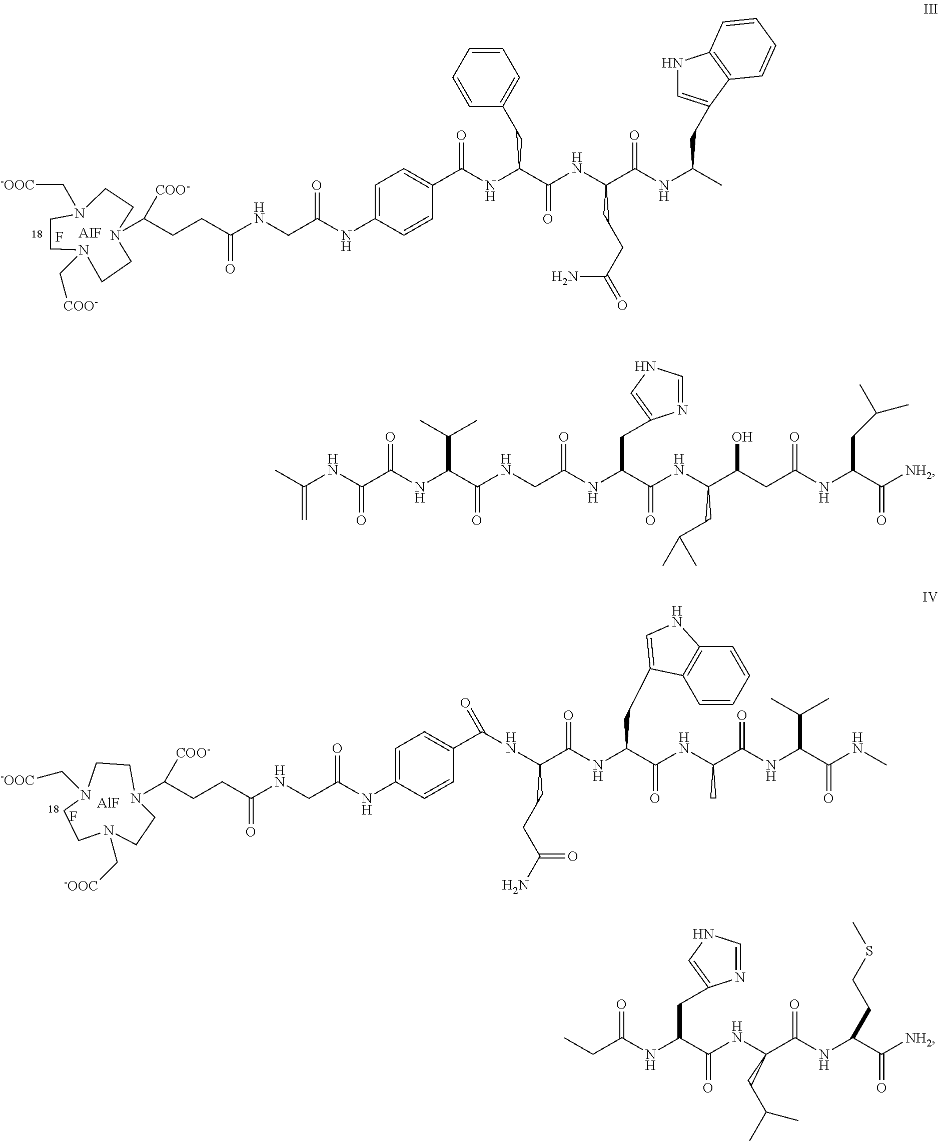

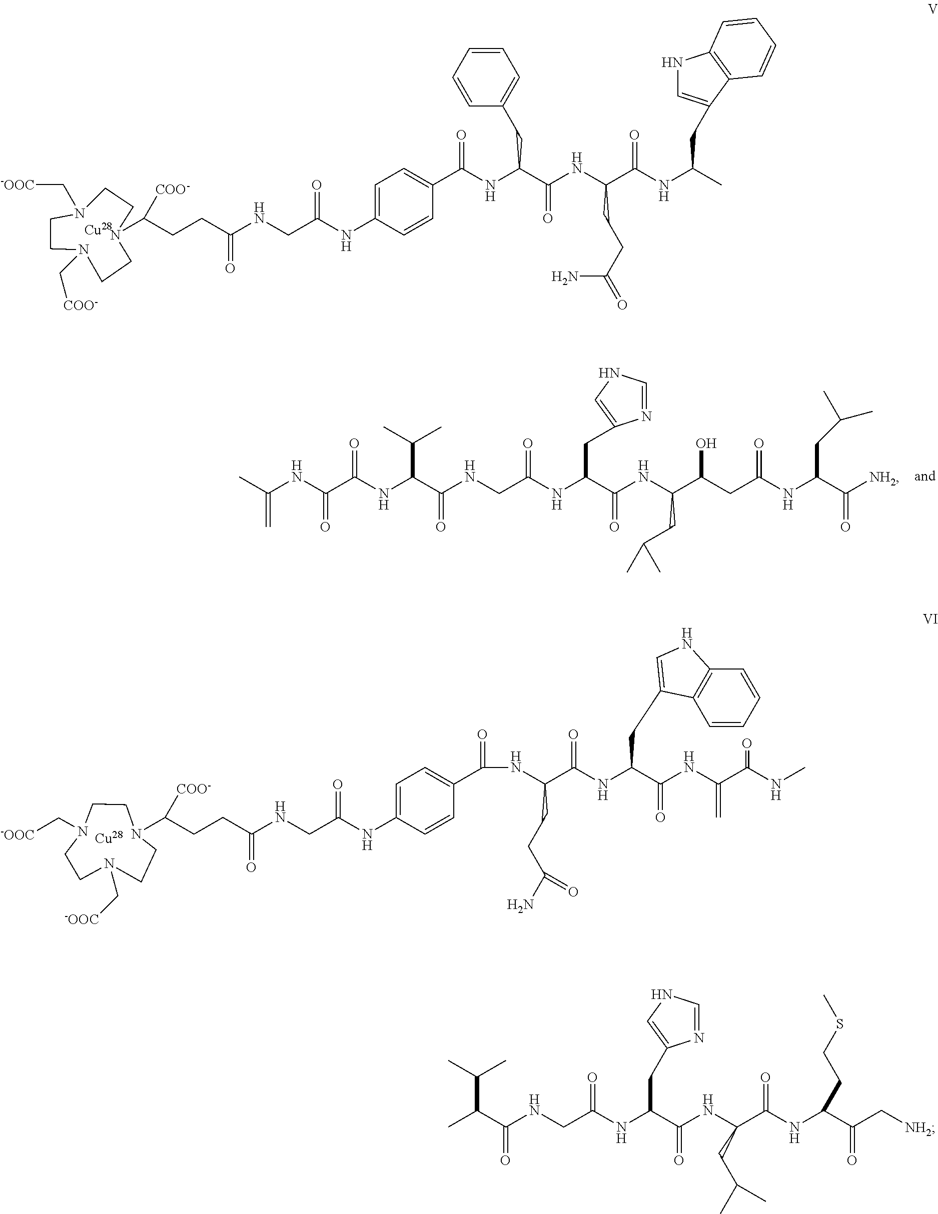

determining the location of the probe in the recipient human or non-human animal; and identifying a tissue in the animal or human host, wherein the amount of the detectable label in the tissue is greater than in other tissues of the host, thereby identifying a population of cancer cells having a gastrin-releasing peptide receptor thereon.

In embodiments of this aspect of the disclosure, the gastrin-releasing peptide receptor-probe can be detected by positron emission tomography scanning.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

Now having described the embodiments of the disclosure, in general, the example describes some additional embodiments. While embodiments of present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### EXAMPLES

### Example 1

### Material and Methods;

All of the chemicals obtained commercially were of analytic grade and used without further purification. The PC3 human prostate carcinoma cell line was obtained from the American Type Culture Collection (Manassas, Va., USA). Male nude mice were purchased from the Charles River Laboratory (Wilmington, Mass., USA). ⁶⁴Cu was provided by the Department of Medical Physics, University of Wisconsin at Madison (Madison, Wis., USA). No-carrier-added 18F was obtained from an in-house PETtrace cyclotron (GE Healthcare). The Sep-Pak C18 cartridges were obtained from Waters (Milford, Mass., USA). The syringe filters and polyethersulfone membranes (pore size, 0.22 μm; diameter, 13 mm) were obtained from Nalgene Nunc International (Rochester, N.Y., USA). 125I-[Tyr4]BBN was purchased from PerkinElmer (Piscataway, N.J., USA). 2,2′-(7-(1-carboxy-4-((2,5-dioxopyrrolidin-1-yl)oxy)-4-oxobutyl)-1,4,7-triazonane-1,4-diyl)diacetic acid (NOTAGA-NHS) was purchased from CheMatech (Dijon, France).

Reverse phase high-performance liquid chromatography (RP-HPLC) was performed using a Dionex 680 chromatography system with a UVD 170U absorbance detector (Sunnyvale, Calif., USA) and 105S single-channel model radiation detector (Carroll & Ramsey Associates). A Vydac protein and peptide column (218TP510; C18, 5 μm, 250×10 mm) was used for semi-preparative HPLC at a flow rate of 4 mL/min. The mobile phase was maintained at a constant 95% for solvent A (0.1% trifluoroacetic acid (TFA) in water) and 5% for solvent B (0.1% TFA in acetonitrile (MeCN)) at 0-5 min and subsequently changed to 35% for solvent A and 65% for solvent B at 35 min. A Vydac protein and peptide column (218TP510; C18, 5 μm, 250×4.6 mm) was used for the analytical HPLC at a flow rate of 1 mL/min. The mobile phase was changed from an initial 95% for solvent A and 5% for B (from 0-2 min) to 35% for solvent A and 65% for solvent B at 32 min. The recorded data were processed using Chromeleon software (version 6.50) (Sunnyvale, Calif., USA). The UV absorbance was monitored at 218 nm, and the identification of the peptides was confirmed based on the UV spectrum using a PDA detector.

### Example 2

### Chemistry and Radiochemistry

### Preparation of NODAGA-RM1 and NODAGA-AMBA:

The G-4-aminobenzoyl-Gln-Trp-Ala-Val-Gly-His-Leu-Met-NH₂ (AMBA) and G-4-aminobenzoyl-D-Phe-Gln-Trp-Ala-Val-Gly-His-Sta-Leu-NH₂ (RM1) peptides as shown in FIG. 1 were synthesized on a CS Bio CS036A Peptide Synthesizer (Menlo Park, Calif.) using Fmoc-based SPPS as previously described in Miao et al., (2012) J. Nucl. Med. 53: 1110-1118, incorporated herein by reference in its entirety. The peptide purity and molecular masses were determined by analytical scale RP-HPLC and matrix-assisted laser desorption/ionization-time of flight mass spectrometry (MALDI-TOF-MS), respectively. Briefly, Rink amide resin was swollen in N,N-dimethylformamide (DMF) for 30 min. The Fmoc groups were removed using 20% piperidine in DMF. Aliquots of amino acids (1 mmol) were activated in a solution containing 1 mmol hydroxybenzotriazole (HOBt) and 0.5 M diisopropylcarbodiimide (DIC) in DMF. Following synthesis, side-chain deprotection and resin cleavage were achieved by the addition of a 92.5:2.5:2.5:2.5 (v/v) mixture of TFA:triisopropylsilane:ethanedithiol:water for 2-4 h at room temperature. Semi-preparative RP-HPLC was used for the purification.

The two peptides were conjugated with NODAGA-NHS to obtain NODAGA-AMBA and NODAGA-RM1, respectively. Specifically, the peptides (1 μmol each) and NODAGA-NHS (1 μmol) were dissolved in 50 μL of DMF, to which 1 μL of DIPEA was added. The reaction mixtures were stirred for 2 h at room temperature, followed by purification of the final products by the semi-preparative HPLC.

### Example 3

### ⁶⁴Cu and ¹⁸F Labeling:

The ⁶⁴Cu-labeling was performed as previously by Jiang et al., (2010) J. Nucl. Med. 51: 251-258, incorporated herein by reference in its entirety. Briefly, NODAGA-RM1 or NODAGA-AMBA (2 nmol each) was dissolved in NaOAc buffer (0.1 M, pH 5.0) and labeled with 64Cu (2 mCi, 74 MBq) for 60 min at 37° C. The labeled peptides were then purified by analytical HPLC. The radioactive peaks containing the desired products were collected and rotary evaporated to remove the solvent. The products were then formulated in phosphate-buffered saline (1×PBS, pH 7.4) and passed through a 0.22-μm Millipore filter into a sterile vial for the subsequent in vitro and in vivo experiments. The labeling yield was above 90% for all of the probes.

The ¹⁸F—AlF-labeling was performed as previously described in Liu et al., (2011) Eur. J. Nucl. Med. Mol Imaging 38: 1732-1741, incorporated herein by reference in its entirety. A QMA SepPak Light cartridge (Waters) fixed with ¹⁸F (30 mCi, 1.1 GBq) was washed with 2.5 mL of metal-free water. The ¹⁸F was then eluted from the cartridge with 400 μL of 0.4 M potassium bicarbonate from which 200 μL-fractions were taken. The pH of the solution was adjusted to about 4.0 with metal-free glacial acetic acid. Aluminum chloride (2 mM, 3 μL) in 0.1 M sodium acetate buffer (pH 4) and 10 μL of peptide (1 mg/mL in DMSO) were then added to the reaction solution sequentially. The reaction mixtures were incubated at 100° C. for 15 min. The labeled peptides were then purified by semi-preparative HPLC. The fractions containing the desired products were collected and rotary evaporated to remove the solvent. The products were reconstituted in PBS and passed through a 0.22-μm Millipore filter into sterile vials for the subsequent in vitro and in vivo experiments.

### Example 4

### Cell Culture and Cell-Binding Assays:

The PC3 cells were cultured in RM1640 containing high glucose (GIBCO, Carlsbad, Calif.), 10% fetal bovine serum (FBS) and 1% penicillin-streptomycin. The cells were expanded in tissue culture dishes and maintained in a humidified atmosphere of 5% CO₂ at 37° C. The medium was changed every other day. Confluent monolayers were detached using 0.05% trypsin-EDTA and 0.01 M PBS (pH 7.4) and dissociated into a single-cell suspension for further cell culture.

The cell-binding assay was performed similarly as previously reported (18, 35). Briefly, the PC3 cells (3×10⁴) were incubated with 0.06 nM ¹²⁵I-[Tyr4]BBN and varying concentrations of peptides in the binding buffer (RPMI 1640+2 mg/mL BSA+5.2 mg/mL HEPES) at 37° C. for 1 h. The cell-bound, residual radioactivity after washing was determined by gamma counting. The IC50 values, the concentration of competitor required to inhibit 50% of the radioligand binding, were determined by non-linear regression using GraphPad Prism (GraphPad Software, Inc.). The experiments were performed in quadruplicate.

### Example 5

### Mouse Serum Stability:

The in vitro stability of the PET probes was evaluated by incubation with mouse serum (1 mL) at 37° C. for 1 h. The solutions were filtered using a NanoSep 10 K centrifuge (Pall Corp.) to isolate low-molecular-weight radiocomplexes. The samples were analyzed by the radio-HPLC, and the percentages of intact PET probe were determined by quantifying the peaks corresponding with the intact probe and degradation products.

### Example 6

### Small Animal PET Imaging:

Small animal PET scans were performed using a microPET R4 rodent model scanner (Siemens Medical Solutions USA, Inc., Knoxyille, Tenn.). The scanner has a computer-controlled bed and 10.8-cm transaxial and 8-cm axial fields of view (FOVs). Approximately 3×10⁶ cultured PC3 cells were suspended in PBS and subcutaneously implanted in one shoulder of male nude mice. The tumors were allowed to grow to a diameter of 0.6-1 cm (5-6 weeks).

The PC3 xenograft-bearing mice were injected with approximately 1.85 MBq (50 μCi) of either ⁶⁴Cu-NODAGA-RM1 or ⁶⁴Cu-NODAGA-AMBA via the tail vein (n=4 for each group). At the indicated times post-injection (p.i.) (0.5, 1.5 and 4 h), the mice were anesthetized with isoflurane (5% for induction and 2% for maintenance in 100% O₂) using a knock down box. Five-minute static scans were then obtained. The PC3 xenograft-bearing mice were injected with 0.37 MBq (10 Ci) of either ¹⁸F—AlF-NODAGA-RM1 or ¹⁸F—AlF-NODAGA-AMBA probe via the tail vein (n=5 for each group). Blocking studies were performed via tail vein injection of the 18F-probe with cold AMBA (10 mg/kg body weight) (n=5). At 0.5, 1 and 2 h p.i., the small animal PET images were obtained.

The small animal PET images were reconstructed using the two-dimensional ordered-subsets expectation maximization (OSEM) algorithm. No background correction was performed. The region of interests (ROIs; five pixels for coronal and transaxial slices) were designated over the tumor on decay-corrected whole-body coronal images. The maximum counts per pixel per minute were obtained from the ROIs and converted to counts per milliliter per minute by using a calibration constant. Based on an assumed tissue density of 1 g/mL, the ROIs were converted to counts per gram per min. The image ROI-derived percentage of the injected radioactive doses per gram of tissue (% ID/g) values were determined by dividing counts per gram per minute by injected dose. No attenuation correction was performed.

### Example 7

### Animal Biodistribution Studies:

The PC3 xenograft-bearing nude mice (n=5 for each group) were injected with approximately 0.37 MBq (10 Ci) of ¹⁸F—AlF-NODAGA-RM1 via the tail vein and sacrificed at 2 h p.i. The ¹⁸F—AlF-NODAGA-RM1 blocking study was performed by co-injection of the probe with the AMBA peptide (10 mg/kg body weight) via the tail vein. The tumoral and normal tissues of interest were removed and weighed, and their levels of radioactivity were measured using a gamma-counter. The uptake of radioactivity in the tumoral and normal tissues was expressed as a % ID/g.

### Example 8

### Statistical Analysis:

The quantitative data are expressed as the mean values±standard deviation (SD). The mean values were compared using a one-way ANOVA and Student's t test. P-values <0.05 were considered statistically significant.

### Example 9

### Chemistry and Radiochemistry:

The G-4-aminobenzoyl-Gln-Trp-Ala-Val-Gly-His-Leu-Met-NH₂ (chemical formula for [M+H]+: C₆₄H₈₈N₁₆O⁺; calculated MW, 1289.7 Da; measured MW, 1289.6 Da) and G-4-aminobenzoyl-D-Phe-Gln-Trp-Ala-Val-Gly-His-Sta-Leu-NH₂ (chemical formula for [M+H]+: C₅₃H₇₃N₁₅O₁₁⁺; calculated MW, 1116.5 Da; measured MW, 1116.5 Da) peptides were synthesized successfully using a solid phase peptide synthesizer. The NODAGA-peptides were then prepared by direct conjugation of NODAGA-NHS with AMBA or RM1, resulting in 80% yield and >90% purity. Both HPLC and mass spectroscopy were used to confirm the identity of the product. The retention time (Rt) of the purified NODAGA-RM1 was 25.6 min, and the molecular mass was measured as 1646.8 Da for [M+H]+ (chemical formula: C₇₉H₁₁₂N₁₉O₂₀, calculated MW: 1646.8 Da). The Rt of NODAGA-AMBA was 21.2 min, and the m/z was measured as 1473.7 Da for [M+H]+(chemical formula: C₆₇H₉₇N₁₈O₁₈S, calculated MW: 1473.7 Da).

⁶⁴Cu-NODAGA-RM1 and ⁶⁴Cu-NODAGA-AMBA were synthesized in high yield (>85%), and their specific activities were calculated as greater than 800 mCi/μmol. The radiosynthesis of the ¹⁸F—AlF-NODAGA-RM1 and ¹⁸F—AlF-NODAGA-AMBA was completed within 40 min, with decay-corrected yields of 5.6±1.1% and 4.9±1.3%, respectively, and the radiochemical purity of the probes was more than 95%. The specific activities of the purified ¹⁸F—AlF-NODAGA-peptides were calculated as greater than 50 mCi/μmol.

### Example 10

### Cell-Binding Affinity Assay:

The receptor-binding affinity assay results for the RM1, NODAGA-RM1, AMBA and NODAGA-AMBA probes are shown in FIG. 2. All of these peptides inhibited the GRPR-binding of ¹²⁵I-[Tyr4]BBN on the PC3 cells in a concentration-dependent manner. The IC50 values for RM1, NODAGA-RM1, AMBA and NODAGA-AMBA were 0.25±0.04, 4.6±1.0, 0.17±0.07 and 1.9±0.50 nM (n=4), respectively. These results indicate that the incorporation of the NODAGA motif reduced the GRPR-binding affinity of the original peptides but that the resulting bioconjugates still possessed significantly high affinities.

### Example 11

### Serum Stability of the Radiolabeled NODAGA-Peptides:

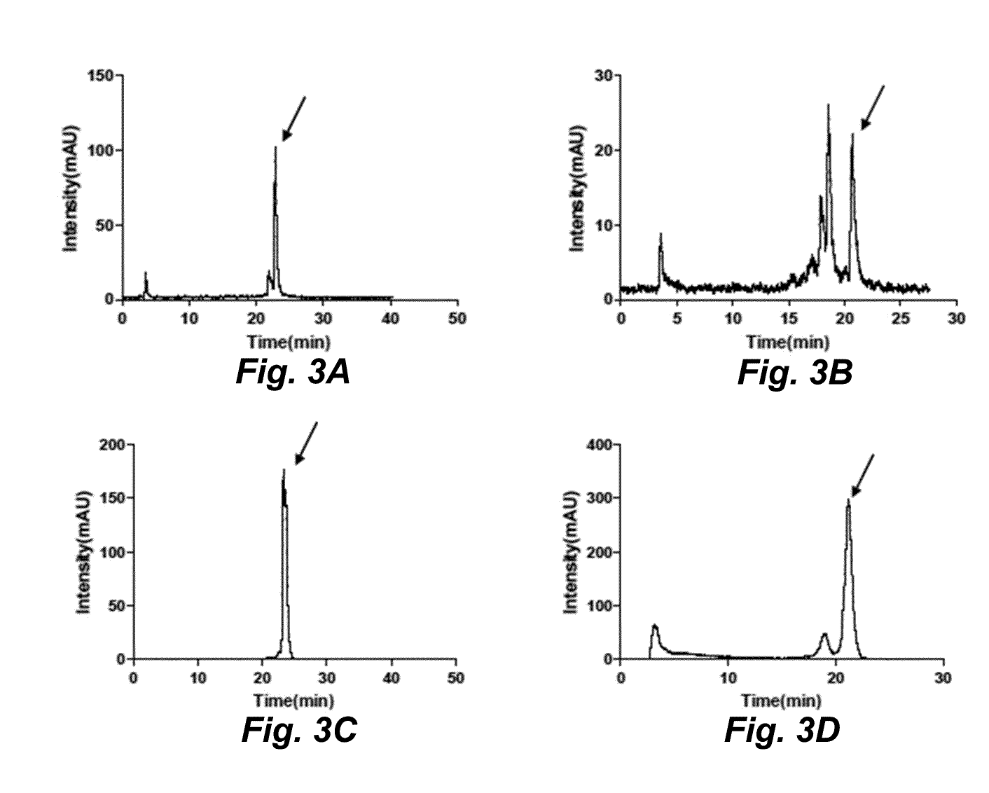

Both ⁶⁴Cu-NODAGA-RM1 and ¹⁸F—AlF-NODAGA-RM1 exhibited favorable stability in mouse serum (as shown in FIGS. 3A and 3C). More than 95% of the probes remained intact after 1 h of incubation in mouse serum at 37° C. However, ⁶⁴Cu-NODAGA-AMBA and ¹⁸F—AlF-NODAGA-AMBA exhibited degradation under the same conditions, as shown in FIGS. 3B and 3D).

### Example 12

### Small Animal PET Imaging Studies:

Representative decay-corrected coronal images of static scans at different time points after injection are shown in FIGS. 4-6. The PC3 tumors were clearly visualized using all of the PET probes. The PC3 tumors were visualized using ⁶⁴Cu-NODAGA-RM1 and ⁶⁴Cu-NODAGA-AMBA with favorable tumor-to-background contrast, even at 0.5 h (as shown in FIG. 4A). Interestingly, ⁶⁴Cu-NODAGA-RM1 exhibited persistent accumulation within the tumor until 4 h p.i., whereas ⁶⁴Cu-NODAGA-AMBA exhibited more rapid tumor clearance and significantly reduced signal within the tumor at 4 h p.i. Further image quantification analysis confirmed the visualization results. The uptake of ⁶⁴Cu-NODAGA-RM1 within the PC3 tumors was 3.3±0.38, 3.0±0.76 and 3.5±1.0% ID/g at 0.5, 1.5 and 4 h p.i., respectively.

In contrast, ⁶⁴Cu-NODAGA-AMBA exhibited only 3.2±0.60, 2.2±0.33 and 1.8±0.10% ID/g tumor uptake at 0.5, 1.5 and 4 h p.i., respectively (as shown in FIGS. 4B and 4C). Moreover, both PET probes exhibited significant accumulation in both the liver and kidneys (>10% ID/g), indicating their clearance through both the hepatobiliary and renal systems. Quantification analysis also revealed that ⁶⁴Cu-NODAGA-RM1 exhibited significantly reduced kidney uptake at 0.5 and 1.5 h compared with ⁶⁴Cu-NODAGA-AMBA (P<0.05), whereas no significant differences were observed between their levels of either kidney uptake at 4 h p.i. or liver uptake at all of the time points as shown in FIGS. 4B and 4C). Increased tumor-to-normal tissue ratios (tumor/liver, tumor/kidney and tumor muscle) were observed for 64Cu-NODAGA-RM1 compared with those for ⁶⁴Cu-NODAGA-AMBA (as shown in FIG. 4D). Overall, ⁶⁴Cu-NODAGA-RM1 exhibited superior in vivo performance compared with the ⁶⁴Cu-NODAGA-AMBA probe.

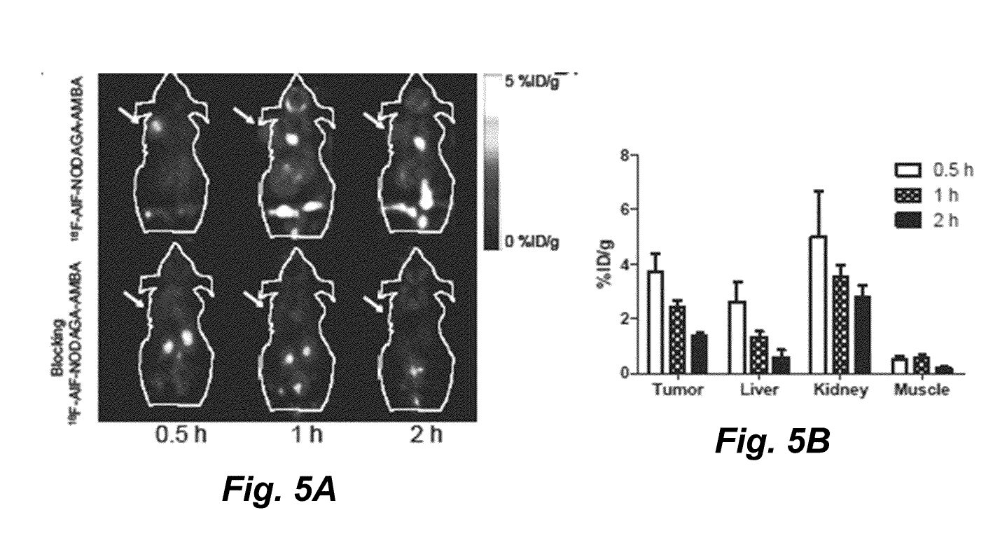

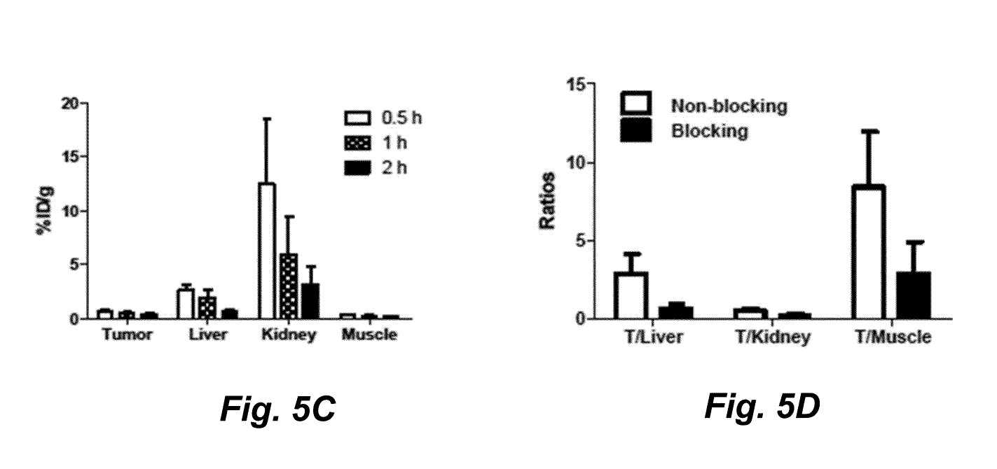

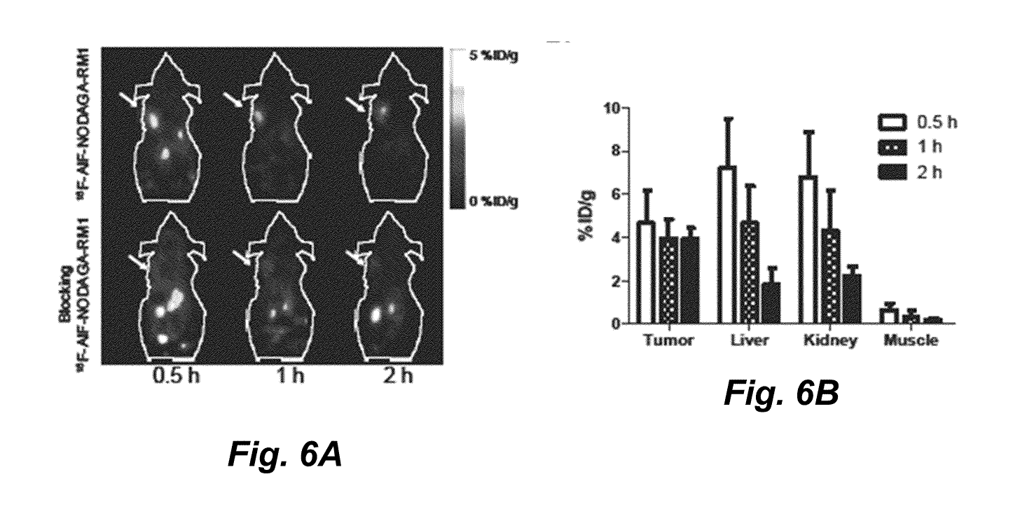

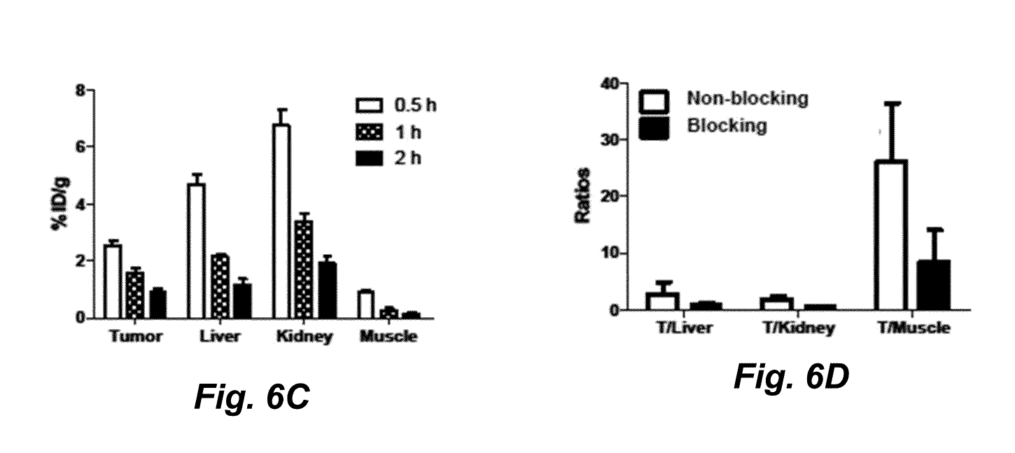

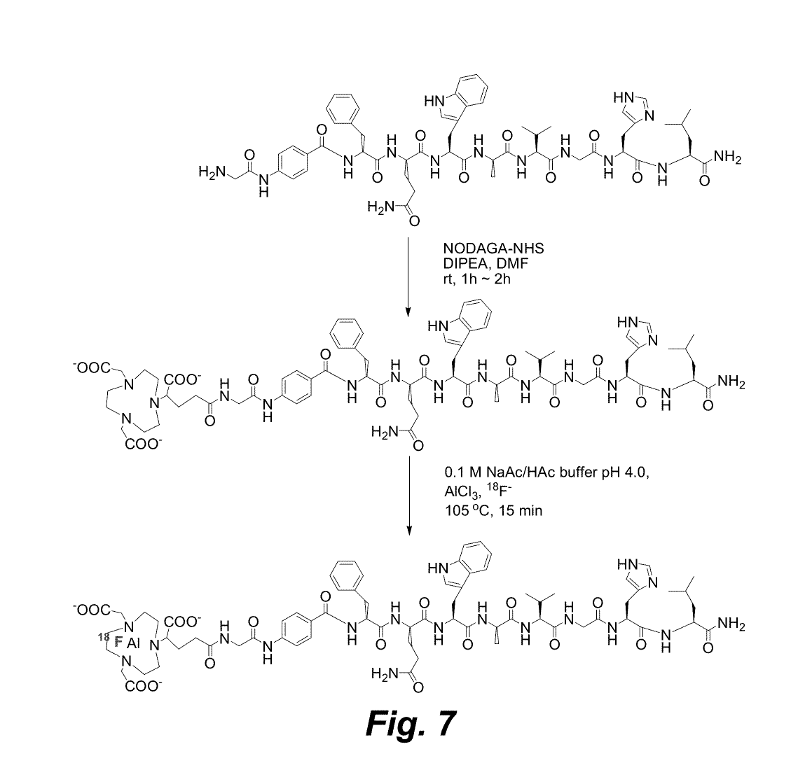

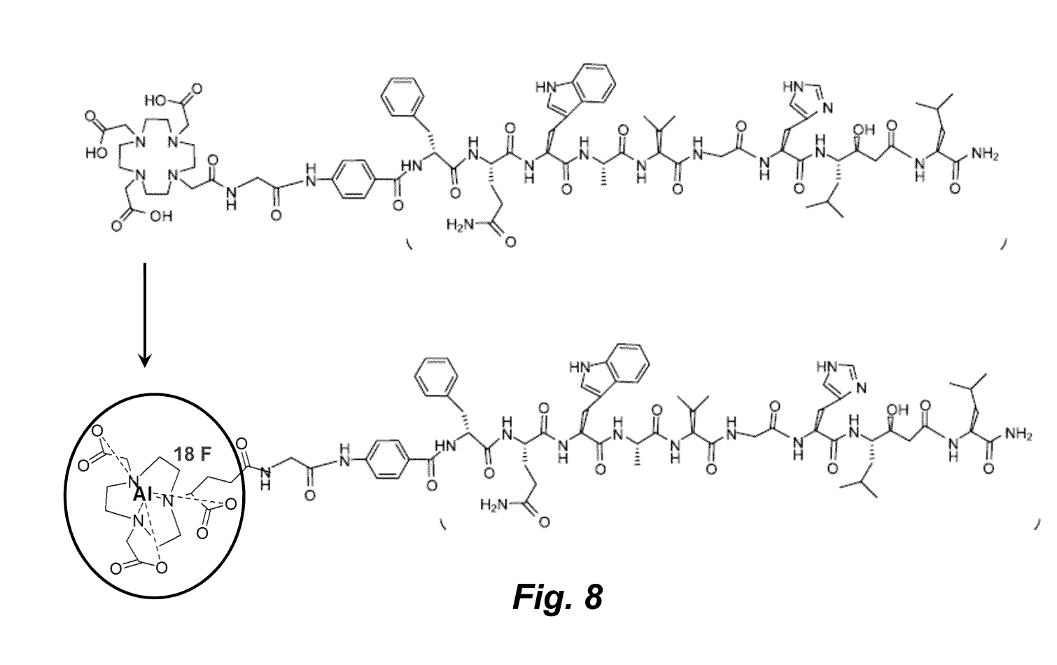

The small animal PET images and quantification analyses for ¹⁸F—AlF-NODAGA-AMBA and ¹⁸F—AlF-NODAGA-RM1 are shown in FIGS. 5 and 6, respectively. As clearly shown in the PET images (FIGS. 5A and 6A, top row), both probes accumulated rapidly within the tumor, and favorable tumor-to-background imaging contrasts were achieved at 0.5 h p.i.

Furthermore, the co-injection of the unlabeled GRPR-targeting peptide AMBA significantly reduced the uptake of both probes (P<0.05), resulting in significantly reduced tumor-to-background contrast in vivo (as shown in FIGS. 5A and 6A, bottom row). These results demonstrated the advantageous in vivo targeting ability and specificity of both probes. Moreover, similar to the ⁶⁴Cu-labeled peptides, ¹⁸F—AlF-NODAGA-AMBA exhibited more rapid tumor clearance than ¹⁸F—AlF-NODAGA-RM1 (FIGS. 5A and 6A, top row). Quantification analysis indicated that the tumor uptake of ¹⁸F—AlF-NODAGA-AMBA was 3.7±0.70, 2.4±0.24 and 1.4±0.13% ID/g at 0.5, 1 and 2 h, respectively (FIG. 5B), whereas the tumor uptake of ¹⁸F—AlF-NODAGA-RM1 was 4.6±1.5, 4.0±0.87 and 3.9±0.48% ID/g at 0.5, 1 and 2 h, respectively (FIG. 6B).

At 1 and 2 h p.i., the tumor uptake of ¹⁸F—AlF-NODAGA-RM1 was significantly increased compared with that of ¹⁸F—AlF-NODAGA-AMBA (P<0.05). The co-injection of ¹⁸F—AlF-NODAGA-AMBA or ¹⁸F—AlF-NODAGA-RM1 with blocking doses of AMBA also significantly reduced their tumor uptake, as shown in FIGS. 5B and 6B. The tumor uptakes were 0.66±0.19, 0.55±0.15 and 0.39±0.18% ID/g for ¹⁸F—AlF-NODAGA-AMBA and 2.5±0.36, 1.6±0.34 and 0.93±0.14% ID/g for ¹⁸F—AlF-NODAGA-RM1 at 0.5, 1 and 2 h p.i., respectively. Lastly, consistent with their corresponding 64Cu-labeled counterparts, both ¹⁸F—AlF-NODAGA-AMBA and ¹⁸F—AlF-NODAGA-RM1 were excreted through both the hepatobiliary and renal systems.

### Example 13

### Biodistribution Studies:

The results of a biodistribution study of ¹⁸F—AlF-NODAGA-RM1 are shown in Table 1.

TABLE 1
The biodistribution of ¹⁸F-AIF-NODAGA-RM1 with or without AMBA
as a blocking agent (10 mg/kg body weight) in PC3 tumor-bearing
nude mice at 2 h p.i. The data are expressed as the normalized
accumulation of activity in % ID/g ± SD (n = 5).

| | | |
|---|---|---|
| Blood | 0.51 ± 0.24 | 0.57 ± 0.11 |
| Heart | 0.42 ± 0.19 | 0.48 ± 0.09 |
| Lungs | 0.77 ± 0.29 | 2.05 ± 2.⁶⁴ |
| Liver | 2.53 ± 0.96 | 1.87 ± 0.19 |
| Spleen | 0.39 ± 0.15 | 0.61 ± 0.41 |
| Pancreas | 5.10 ± 1.44 | 0.55 ± 0.13 |
| Stomach | 0.86 ± 0.13 | 1.04 ± 0.34 |
| Brain | 0.07 ± 0.02 | 0.10 ± 0.03 |
| Intestine | 1.68 ± 1.13 | 3.60 ± 1.85 |
| Kidneys | 4.65 ± 1.95 | 7.32 ± 2.09 |
| Skin | 0.95 ± 0.50 | 2.44 ± 2.48 |
| Muscle | 0.36 ± 0.16 | 0.42 ± 0.13 |
| Bone | 1.58 ± 0.52 | 1.68 ± 0.34 |
| Tumor | 5.25 ± 0.84 | 1.86 ± 0.30 |

Without the presence of AMBA peptide as a blocking agent at 2 h p.i., the tumor, liver and kidney uptakes for ¹⁸F—AlF-NODAGA-RM1 (approximately 20 μCi/mouse, n=5) were 5.2±0.84, 3.1±2.2 and 4.6±1.9% ID/g, respectively. In contrast, the same organ uptakes for the blocking group were 1.8±0.30, 1.9±0.19 and 7.3±2.1% ID/g, which is consistent with the PET imaging results. Increased pancreatic uptake in the control tumor mice (5.1±1.4% ID/g), which is in contrast with reduced pancreatic uptake in the blocking mice (0.54±0.13% ID/g), was also observed. Again, the significantly reduced uptake of the probe in the GRPR-positive tissues, including the PC3 tumor and pancreas, confirmed the receptor-targeting specificity of the probes.

We claim:

1. A probe selectively binding to a mammalian gastrin-releasing peptide receptor (GRPR), the probe comprising a bombesin analogue selected from RM1 and AMBA, the chelator 1,4,7-triazacyclononane, 1-glutaric acid-4,7-acetic acid (NODAGA) conjugated thereto, wherein the probe has a formula I or II:

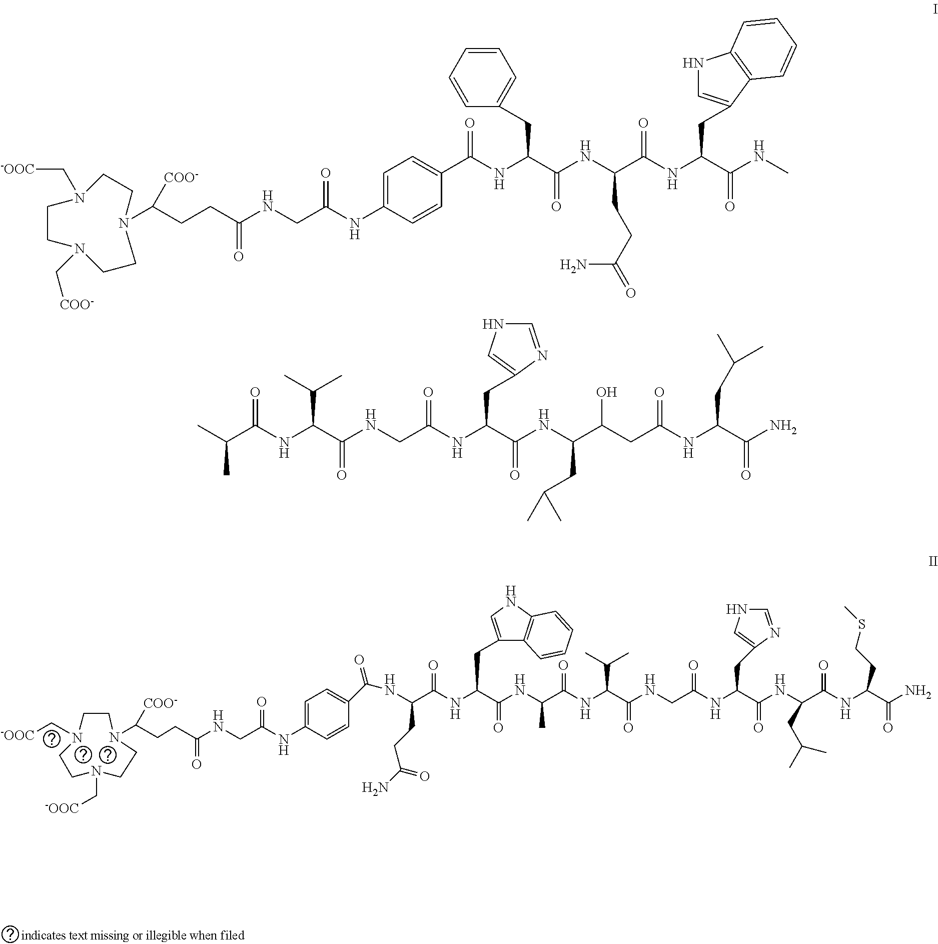

2. The probe of claim 1, further comprising a detectable label, wherein the detectable label is ¹⁸F or ⁶⁴Cu.

3. The probe of claim 2, wherein the probe has a formula selected from the group consisting of formulas III, IV, V, and VI:

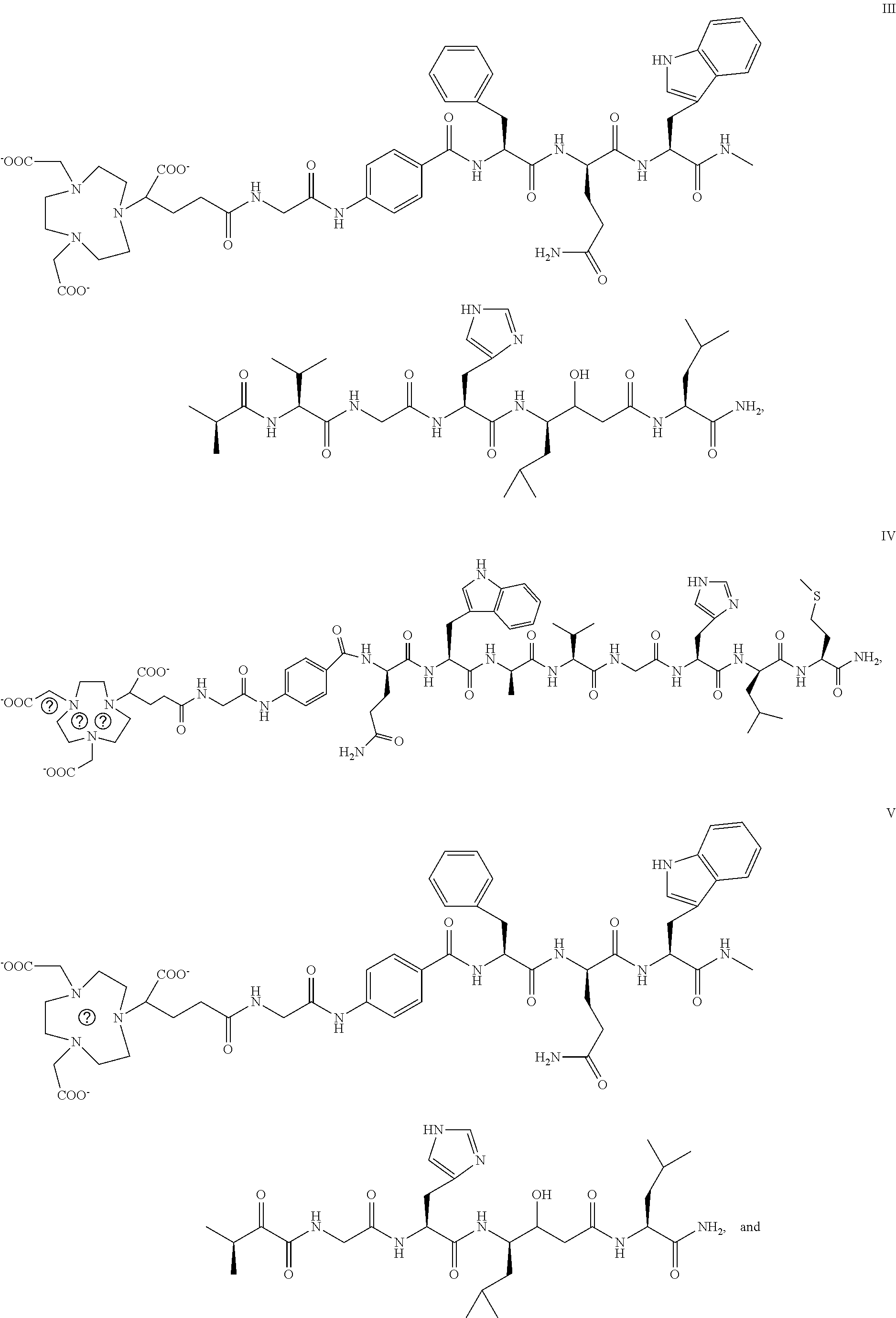

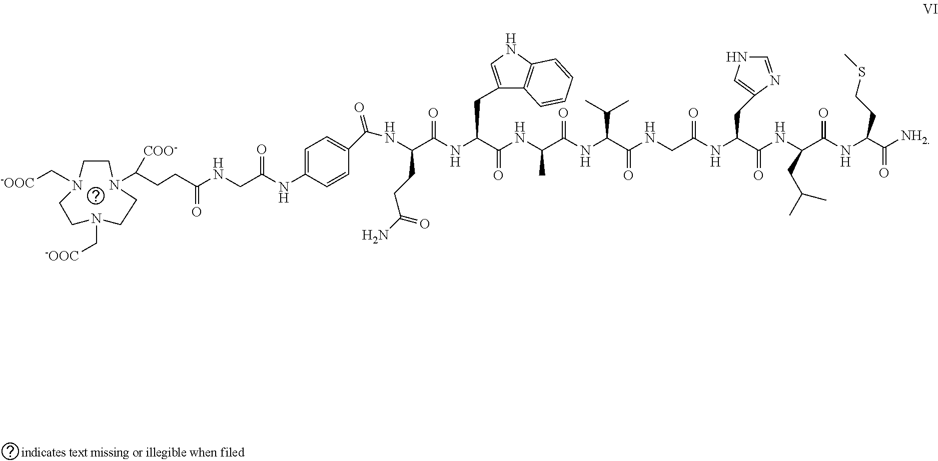

4. The probe of claim 3, wherein the probe has the formula III.

5. The probe of claim 3, wherein the probe has the formula IV.

6. The probe of claim 3, wherein the probe has the formula V.

7. The probe of claim 3, wherein the probe has the formula VI.

8. A pharmaceutically acceptable probe composition comprising at least one probe selected from the group consisting of from formulas III-VI: 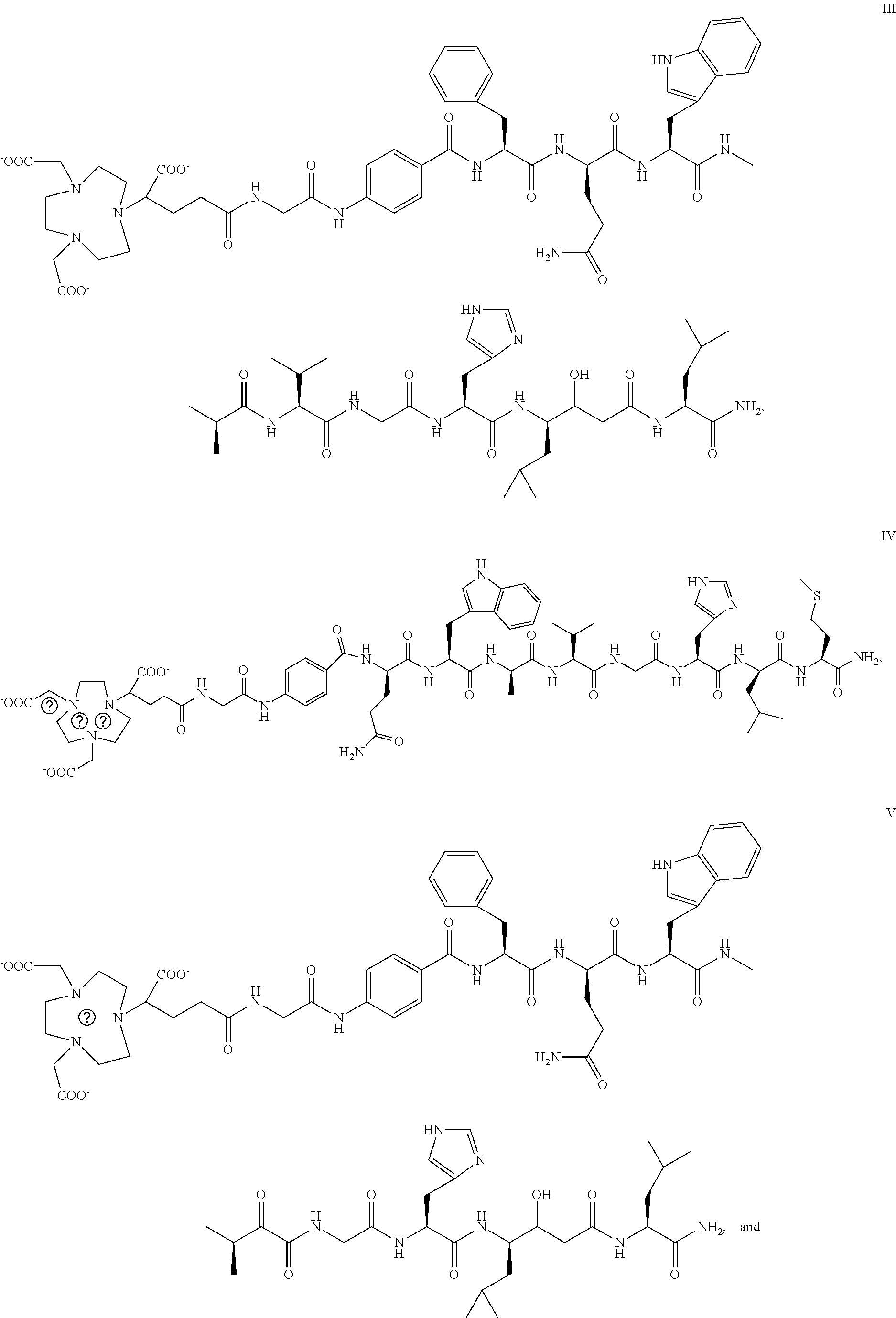 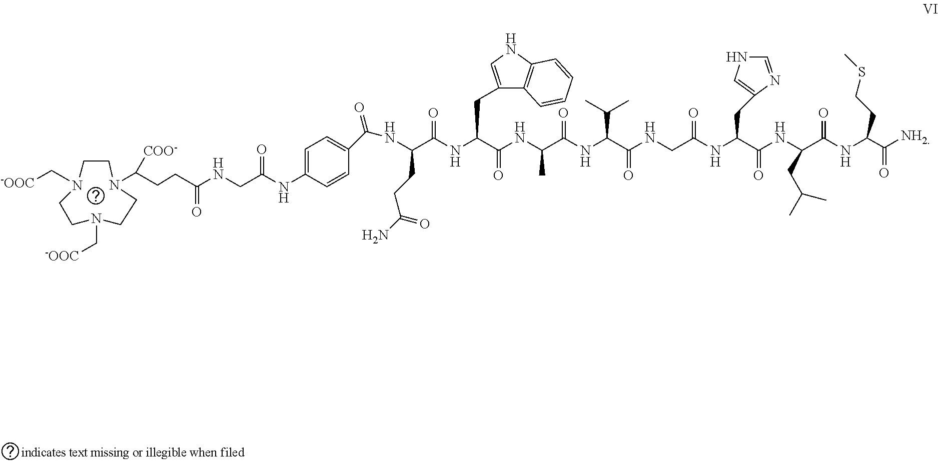
and a pharmaceutically acceptable carrier.

9. A method of identifying a cell or a population of cells expressing a gastrin-releasing peptide receptor, said method comprising:
contacting a cell or population of cells with a composition comprising at least one probe having a radionuclide and selected from the group consisting of the formulas III-VI: 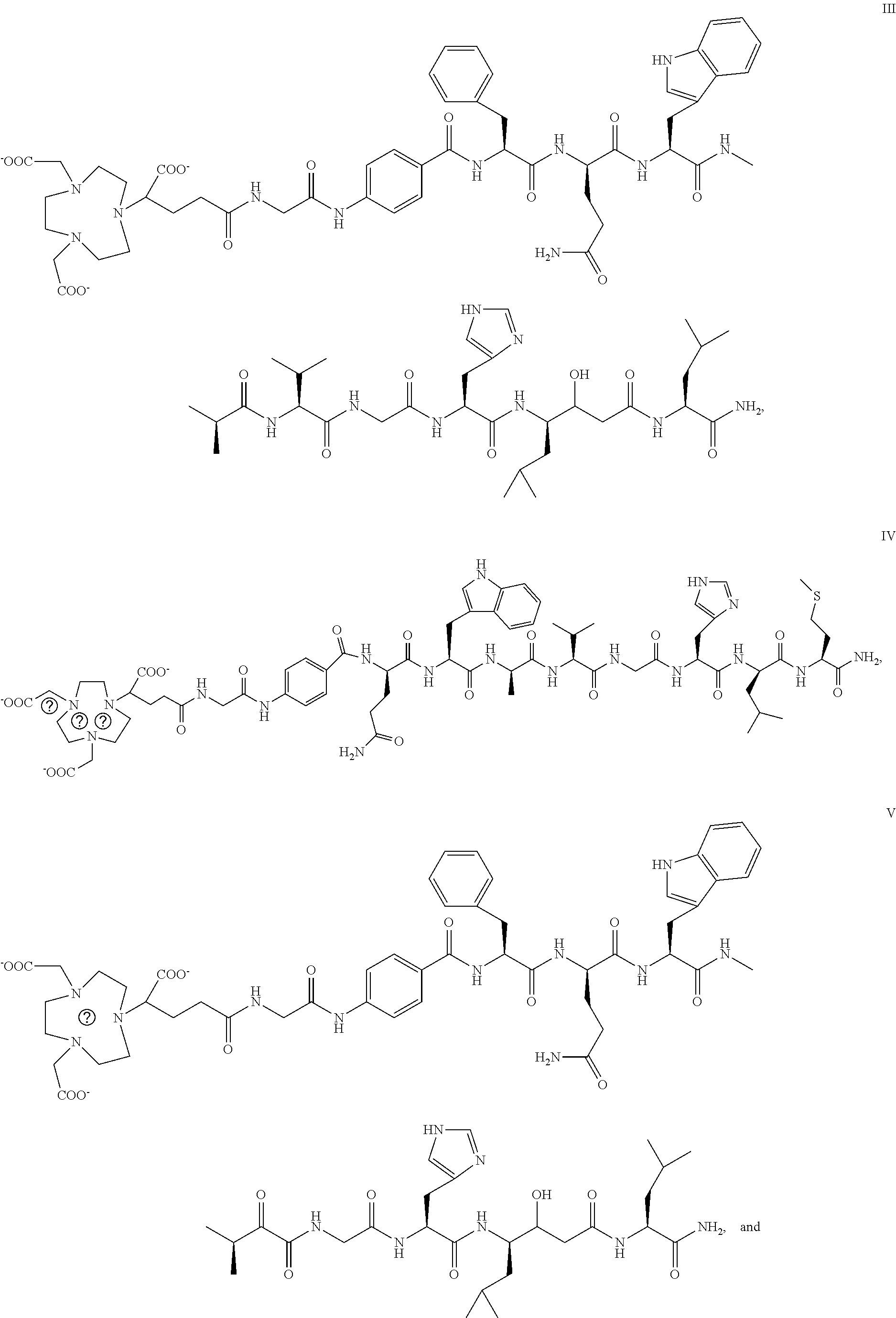 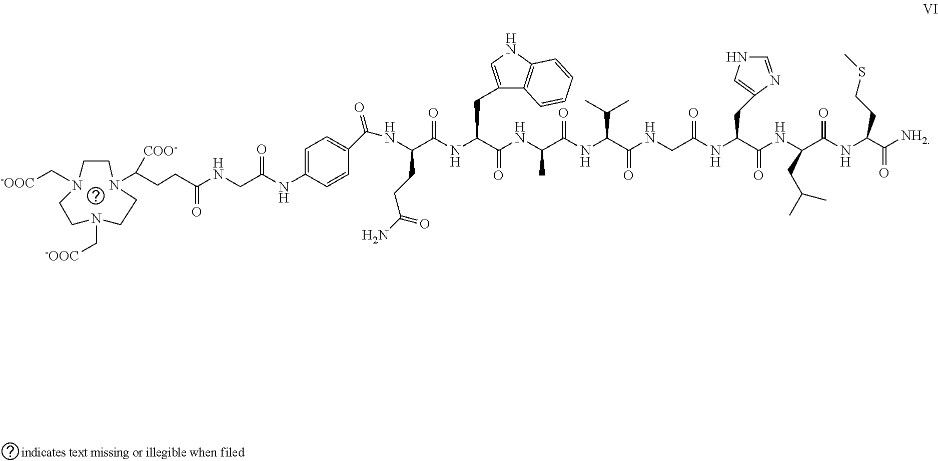
allowing the probe to selectively bind to a gastrin-releasing peptide receptor of a cell or a population of cells; and
detecting the radionuclide presence on the cell or population of cells, whereby the presence of the radionuclide indicates the cell or population of cells has a gastrin-releasing peptide receptor thereon.

10. The method of claim 9, wherein the probe composition further comprises a pharmaceutically acceptable carrier.

11. The method of claim 9, further comprising the step of delivering the probe to a human or non-human animal.

12. A method of detecting in a human or non-human animal a localized population of cancer cells having a gastrin-releasing peptide receptor, said method comprising the steps of:
administering to a human or non-human animal a gastrin-releasing peptide receptor-specific probe selected from the group consisting of the formulas III-VI: 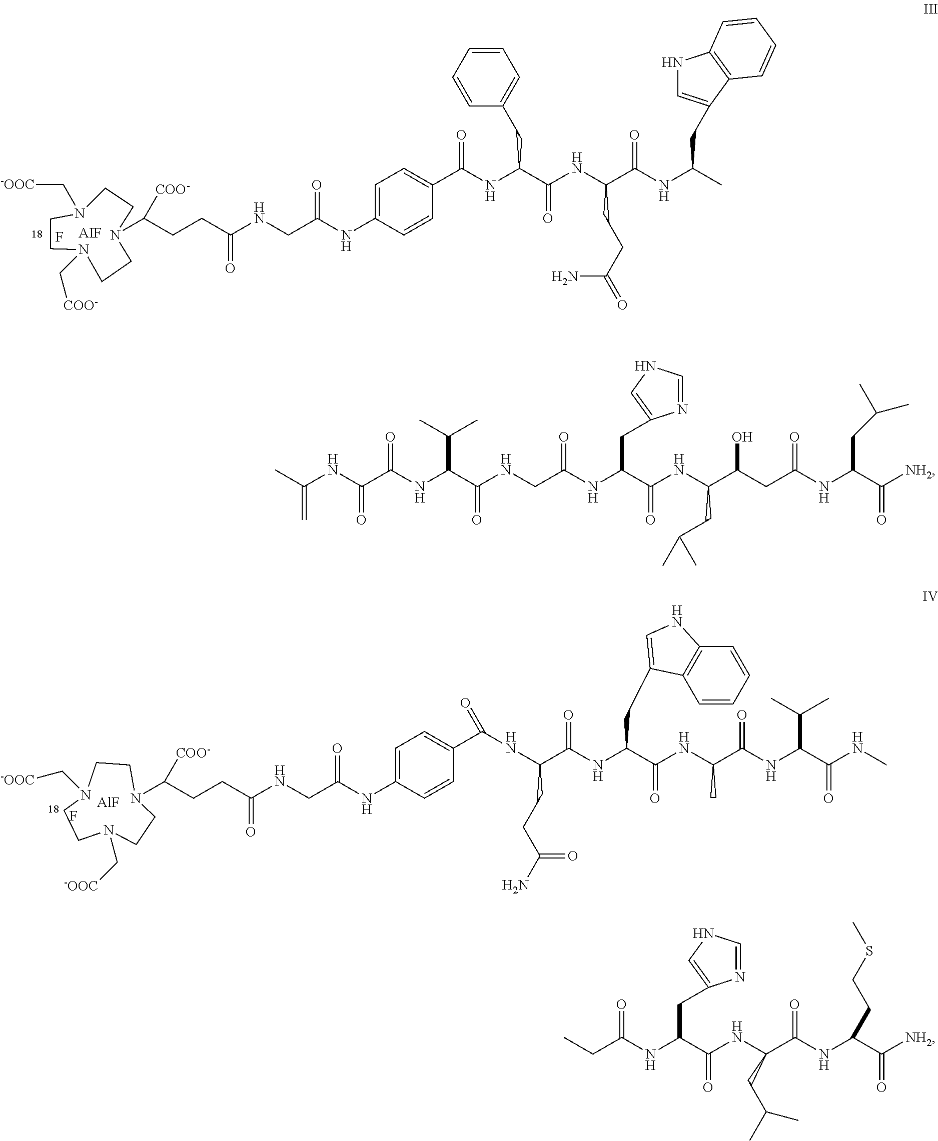 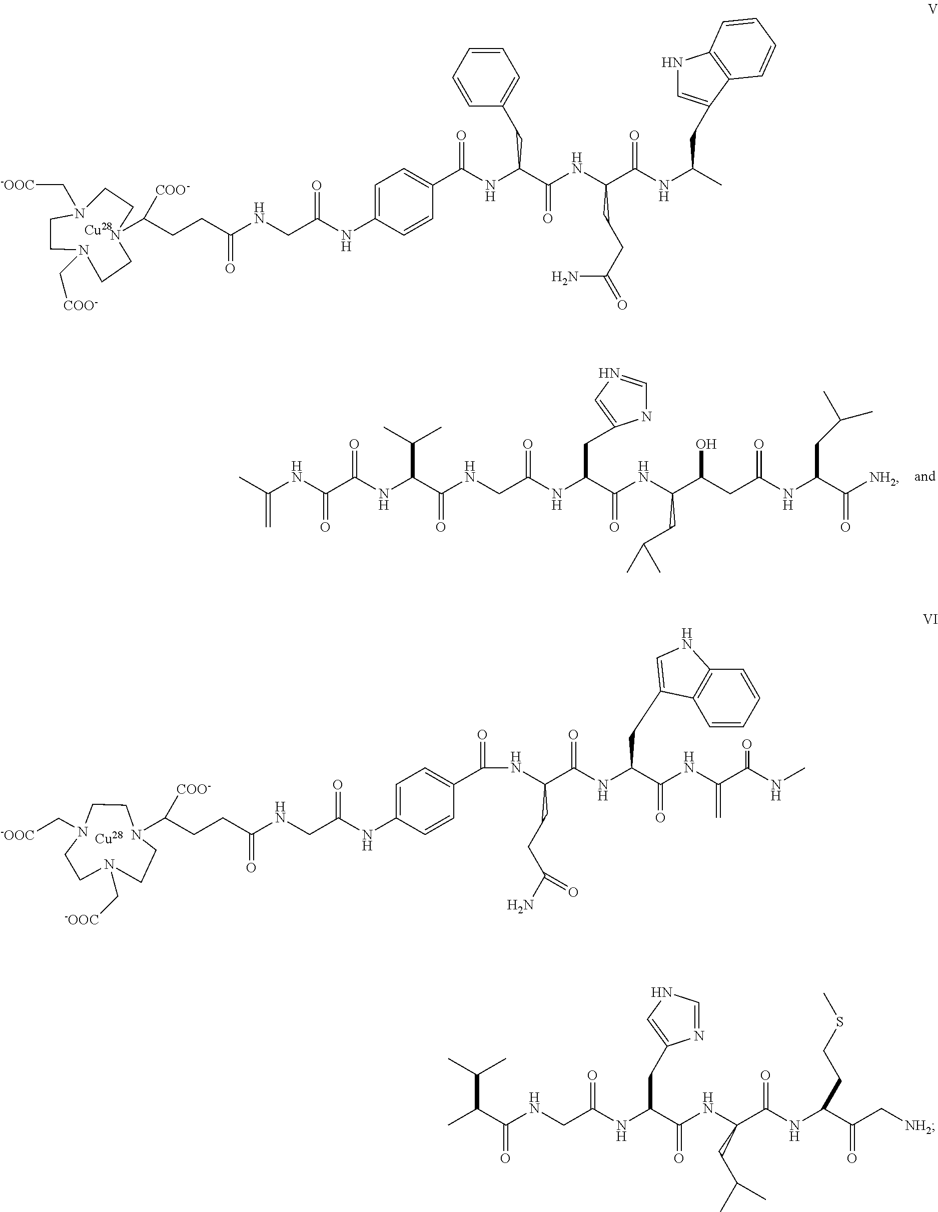
determining the location of the probe in the recipient human or non-human animal;
identifying a tissue in the animal or human host wherein the amount of the detectable label in the tissue is greater than in other tissues of the host, thereby identifying a population of cancer cells having a gastrin-releasing peptide receptor thereon.

13. The method of claim 12, wherein the gastrin-releasing peptide receptor-probe is detected by positron emission tomography scanning.
